(19) United States
(12) Patent Application Publication
(10) Pub. No.: US 2011/0263014 A1
(43) Pub. Date: Oct. 27, 2011

(54) Cleavage of Nanog by Caspases Mediates the Differentiation of Embryonic Stem Cells

(75) Inventors: Thomas P. Zwaka, Houston, TX (US); Jun Fujita, Houston, TX (US); Ana Crane, The Woodlands, TX (US); Marion Dejosez, Houston, TX (US)

(73) Assignee: BAYLOR COLLEGE OF MEDICINE, Houston, TX (US)

(21) Appl. No.: 12/989,593

(22) PCT Filed: May 8, 2009
(86) PCT No.: PCT/US09/43303

§ 371 Date: Apr. 12, 2011

Related U.S. Application Data
(60) Provisional application No. 61/127,090, filed on May 9, 2008.

(51) Int. Cl. C12N 5/071; C12N 5/02

(52) U.S. Cl. 435/366; 435/375; 435/377

(57) ABSTRACT

The present invention is based on the discovery that a caspase specifically cleaves the transcription factor, Nanog, leading to the initiation of cellular differentiation of embryonic stem (ES) cells. The present invention includes a method of inhibiting the cleavage of Nanog, thereby maintaining the pluripotency of an ES cell or preventing the differentiation of an ES cell. The present invention further provides compositions and methods for inhibiting caspase expression, activity, and/or stability.

### BACKGROUND OF THE INVENTION

Embryonic stem (ES) cell research holds remarkable promise, yet the mechanisms by which these cells transition from pluripotency to differentiation have been elusive. It now appears that a small core set of transcription factors work together to maintain the pluripotent state of ES cells (Bernstein et al., 2006, Cell 125:315-326; Boyer et al., 2005, Cell 122:947-956; Boyer et al., 2006, Nature 441:349-353; Lee et al., 2006, Cell 125:301-313). These transcriptional regulators, including Oct4, Sox2 and Nanog, stimulate the expression of genes controlling self-renewal while repressing genes that drive differentiation. An emerging hypothesis is that Nanog and other core transcription factors form a tight, autoregulatory circuit that enables ES cells to remain stable in culture and ensures extreme autonomy in proliferative decisions (Boyer et al., 2005, Cell 122:947-956; Chickarmane et al., 2006, PLoS Comput. Biol. 2:e123). Thus, ES cells depend only marginally on mitogenic stimuli typically required for somatic cells to proliferate, but stimulate their own growth through endogenous factors. This autonomy is best shown by the unique ability of ES cells, injected into virtually any anatomical site in adult animals, to form rapidly growing tumors called teratomas (Damjanov and Solter, 1974, Curr. Topics Pathol. 59:69-130).

The cell death system pathways incorporate a subgroup of cysteine proteases, the so-called terminal executioner caspases (e.g. caspases-3, -6 and -7) (Earnshaw et al., 1999, Ann. Rev. Biochem. 68:383-424; Thornberry and Lazebnik, 1998, Science 281:1312-1316), which cleave several vital proteins leading ultimately to sequential and controlled cellular breakdown (Kidd, 1998, Ann. Rev. Physiol. 60:533-573). In addition, caspase-3 is capable of cleaving and activating other caspases, including caspase-2, that have their own targets. These caspases are very specific for particular amino acid sequences, are highly regulated in their activities, and in some contexts appear to influence the decision of cells to differentiate (Arama et al., 2003, Dev. Cell. 4:687-697; De Bolton et al., 2002, Blood 100:1310-1317; De Maria et al., 1999a, Blood 93:796-803; De Maria et al., 1999b, Nature 401:489-493; Ishizaki et al., 1998, J. Cell Biol. 140:153-158), implying functions other than the execution of cell death programs.

There has been a long standing need in the art to identify the endogenous mechanisms that regulate the induction of ES cell differentiation. The present invention fulfills this need.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1, comprising FIG. 1A through FIG. 1F, is a series of images depicting increased caspase activity in mouse ES cells upon induction of differentiation. FIG. 1A is a graph depicting caspase 3 activity at different time points following stimulation with RA. The R1 ES cell line was stimulated with retinoic acid (RA) (1 μM) for the indicated times and caspase activity was measured in an in vitro caspase activity assay. The data are means±standard deviation (SD) of triplicate experiments. FIG. 1B is a graph depicting the percentage of cells undergoing programmed cell death determined by counting apoptotic bodies after the same ES cell line was again exposed to RA for various times. UV, ultraviolet light. The data are means±SD. FIG. 1C is a series of images depicting ES cells expressing the caspase sensor (Casp^{sensor}) after they were stimulated with RA (1 μM), fixed at the four indicated time points and stained with an antibody against a reporter protein (enhanced yellow fluorecent protein, EYFP). Immunofluorescence images (40×) were taken from representative fields. Although a shift of the signal from the cytoplasm to the nucleus is apparent at 12 hours, the cells did not show any signs of apoptosis. Cells treated with stausporine at 6 hours served as positive controls; scale bar=10 μm. FIG. 1D is a graph depicting the mean percentage of cells with mainly cytoplasmic or nuclear staining determined for a similar experimental setting as in FIG. 1C. FIG. 1E is an image depicting Western blot analysis of nuclear lysates isolated from mouse ES cells after stimulation with RA for the indicated times. The blotted membrane was probed with an antibody against PARP-1. The uncleaved form of PARP-1 is apparent at the earlier poststimulation times, with the cleaved form (85-kDa fragment) appearing after 24 hours, indicating PARP-1 cleavage. FIG. 1F is an image depicting immunostaining of a mouse ES cell colony stimulated with RA or incubated with leukemia inhibitory factor (LIF) for 2 days. The antibody used specifically recognizes the 85 kDa form of PARP-1. Magnification, 40×.

FIG. 2, comprising FIG. 2A through FIG. 2E, is a series of images depicting an increase in caspase-3 during differentiation and that caspase-3 is essential for proper differentiation of ES cells. FIG. 2A is an image depicting Western blot analysis of protein lysates from ES cells stimulated to differentiate with RA for the indicated times. The Western blot membranes were probed with an antibody that specifically recognizes only active caspase-3. MEF, mouse embryonic fibroblasts. FIG. 2B is a series of images depicting alkaline phosphatase staining of ES cell colonies three (3) days after being stimulated with RA (1 μM/ml; middle panel) or co-incubated with RA and the pan-caspase blocking peptide VAD.fmk (100 μM; right panel). Cells incubated with leukemia inhibitory factor (LIF; left panel) served as controls. The majority of colonies lost staining due to differentiation, whereas coincubation with VAD prevented differentiation. Magnification, 10×. FIG. 2C is a series of images depicting Casp3 heterozygous (+/−) and knockout (−/−) ES cells stimulated with RA (1 μM) for 3 days and stained for alkaline phosphatase. Casp3^{+/-} ES cells differentiated, whereas the majority of Casp3^{-/-} ES cells did not (10×). Controls were cells incubated without LIF. FIG. 2D is a pair of graphs depicting (left) the quantification of colonies shown in FIG. 2B as described in Experimental Procedures, and (right) quantification of colonies shown in FIG. 2C. Mean values are shown. FIG. 2E is a pair of images depicting Casp3^{+/-} (left panel) and Casp3^{-/-} (right panel) ES cells injected into immunocompromised nude mice. The resultant tumors were analyzed histologically. Typical aspects of Casp3^{+/-} and of Casp3^{-/-} tumors included derivatives from all three germ layers within a teratoma or immature and undifferentiated cells, respectively.

FIG. 3 comprising FIG. 3A through FIG. 3C, is a series of images depicting ectopic activation of caspase-3 activity in ES cells that leads to differentiation. FIG. 3A is an image depicting Western blotting and detection of Oct4 and Nanog performed after ES cells carrying a constitutively active form of Casp3 gene (Casp3rev) or a mutated version (mCasp3rev) under the control of a tetracycline-inducible element were stimulated with doxycycline at increasing concentrations. Both Oct4 and Nanog disappeared when the expression of Casp3rev but not mCasp3rev increased. WT, wild-type. FIG. 3B is an image depicting ES cells with clear morphologic signs of differentiation when Casp3rev but not mCasp3rev expression is increased. FIG. 3C is an image of a gel depicting the results of RT-PCR of Oct4, Nanog and differentiation markers. Both Oct4 and Nanog were downregulated when expression of Casp3rev but not mCasp3rev was increased, whereas expression of the differentiation markers Bmp2, Laminin B1 and Gata4 increased.

FIG. 4 comprising FIG. 4A through FIG. 4G, is a series of images depicting caspase-3 cleavage of Nanog in ES cells. FIG. 4A is an image depicting cleavage of in vitro-translated hNanog (top), but not its D69E mutant (bottom), by caspase-3. Proposed caspase cleavage site at position 69, separating the N-terminal domain of Nanog from the rest of the protein. FIG. 4B (top) is an image depicting a Western blot showing expression of hNanog and mutant hNanog with or without a mutation at position 69 (D69E) that obviates cleavage. The results represent ES cells transfected with hNanog or D69E hNanog in cultures treated with RA (1 μM) or untreated. After induction of differentiation, cleaved hNanog appeared as a 27 kDa band that was not visible when the cells were transfected with the mutated form of hNanog. FIG. 4B (bottom) is an image depicting a Western blot showing expression of hNanog and mNanog and mutant hNanog and mNanog carrying a mutation at position 69 and 67 respectively (D69E, D67G) that obviates cleavage. The results represent ES cells transfected with depicted Nanog versions and treated with RA. Cleaved hNanog and mNanog appeared as a 27 kDa band that was barely visible when the cells were transfected with the mutated forms of hNanog and mNanog. FIG. 4C is an image depicting Western blot analysis for endogenous mNanog reveals the cleaved form of the protein after induction of differentiation with RA. FIG. 4D is an image depicting in vitro caspase cleavage assay reveals that caspase-9 can effectively cleave in vitro-translated mNanog. FIG. 4E is a graph depicting the quantification of proliferation of ES cells harboring hNanog or D69E hNanog and grown under normal conditions with LIF or RA stimulation. Mean (±SD) levels of ³H incorporation in triplicate experiments indicate a consistently greater proliferative advantage for cells expressing the hNanog cleavage mutant. FIG. 4F is a graph depicting proliferation of ES cells expressing D69E or wild-type hNanog that were mixed 20:80 with EGFP-labeled ES cells. The percentage of unlabeled ES cells in the mixture was determined during growth in the absence of LIF. Cells carrying the caspase cleavage-resistant mutant (D69E hNanog) had a marked growth advantage over control or hNanog-expressing cells. The data are means (±SD) of triplicate experiments. FIG. 4G is a pair of graphs depicting mouse ES cells plated at low densities (5000 cells/cm²) and transfected with wildtype mNanog, D67G mNanog (caspase cleavage-resistant Nanog) or control plasmid (GFP) and cultured with (top) or without LIF (bottom) for 3 days. Transfection with D67G mNanog substantially increased the number of undifferentiated (AP-positive) colonies.

FIG. 5 is a graph depicting increased caspase activity in ES cells upon induction of differentiation. The D3 ES cell line was stimulated with retinoic acid (RA) or N2B27 differentiation medium for the indicated times. Caspase activity was measured with an in vitro assay. Mean (±SD) measurements from triplicate experiments are shown.

FIG. 6, comprising FIG. 6A and FIG. 6B, is a series of images depicting the caspase sensor system. FIG. 6A is a schematic illustration depicting the caspase sensor containing a nuclear translocation signal (NLS), enhanced yellow fluorescent protein (EYFP), a 30-nucleotide-long sequence encoding the caspase cleavage site of PARP-1, and a cytoplasmic translocation signal (NES). This fragment was integrated upstream of the HPRT locus in 17-2lox ES cells via Cre-mediated recombination, placing it under the control of a tetracycline response element (TRE). The tet transactivator (rtTA) is targeted to the rosa26 locus on chromosome 6. FIG. 6B is an image depicting Western blot analysis of protein lysates extracted from cells treated as indicated at the top of the graphic. Only doxycycline-treated cells showed expression of the reporter protein, which was cleaved only after induction of apoptosis.

FIG. 7, comprising FIG. 7A through FIG. 7D, is a series of images depicting the characterization of Casp3 knockout ES cell lines. FIG. 7A is an image depicting genotyping PCR analysis showing that the knockout ES cell lines 34A and 34B contain only the knockout allele (smaller fragment), whereas the cell lines 22A and 22B contain both the larger wild-type allele and the smaller knockout allele. FIG. 7B is an image depicting differentiation of Casp3^{+/-} (right) and Casp3^{-/-} (left) ES cell lines stimulated according to an embryoid body differentiation protocol. Although both cell lines initially formed similar embryoid bodies post-stimulation (day 4), there were clear differences at 8 days. The Casp3^{-/-} ES cells remained largely compacted and showed typical ES cell morphology, whereas Casp3^{+/-} ES cells had differentiated completely. This difference had become even more pronounced by day 12. FIG. 7C is an image depicting quantification of Oct4 activity by semiquantitative reverse transcriptase PCR. On days 8 and 10, Casp3^{-/-} cells had more Oct4 signal than did Casp3^{+/-} cells, indicating delayed differentiation. FIG. 7D is a graph depicting the results obtained from an experiment similar to that shown in FIG. 7C but with real-time quantitative PCR wherein Casp3^{-/-} ES cells (gray line) differentiate more slowly than Casp3^{+/-} ES cells (black line).

FIG. 8 is a series of images depicting the results of a teratoma formation experiment. Casp3^{+/-} teratomas (left panels) form typical structures such as neural epithelium (top) cartilage (middle) and endodermal structures (bottom). Casp3^{-/-} tumors (right panels) are extremely homogenous and contain undifferentiated cell types.

FIG. 9, comprising FIG. 9A through FIG. 9C, is a series of images depicting the inducible caspase-3 system. FIG. 9A is a schematic illustration of the constitutively active Casp3 gene (Casp3rev) and a mutated version carrying a point mutation that renders the gene inactive (mCasp3rev) which were subcloned. These fragments were then integrated upstream of the HPRT locus in A2Lox ES cells via Cre-mediated recombination, placing them under control of a tetracycline response element (TRE). The tet transactivator (rtTA) was targeted into to the Rosa26 locus on chromosome 6. FIG. 9B is an image depicting Western blot analysis of protein lysates extracted from two cell lines stimulated with increasing concentrations of doxycycline. Only doxycycline-treated cells carrying the caspase-3 protein showed increased levels of active caspase-3. FIG. 9C is a graph depicting the results of an experiment where the same two cell lines as shown in FIG. 9A and FIG. 9B were stimulated with doxycycline, and intrinsic caspase activity was measured with an in vitro caspase activity assay. Values are means (±SD) of triplicate experiments.

FIG. 10, comprising FIG. 10A through FIG. 10C, is a series of images depicting cleavage of mNanog by caspases. FIG. 10A is an image depicting in vitro-translated mNanog is not cleaved by caspase-3. FIG. 10B is a graph depicting the quantification of a caspase-3 activity assay (CaspGlo) that reveals that both the hNanog caspase cleavage site peptide (DSPD) and the mNanog caspase cleavage site peptide (GSPD) effectively inhibit caspase-3. FIG. 10C is an image depicting the immunoprecipitation of mNanog and mNanog (D67G) purified from ES cells and exposed to recombinant caspase-3 and caspase-9. Only caspase-9 effectively cleaved mNanog into a 27 kDa fragment. Mutation D67G in mNanog greatly diminished cleavage of mNanog by caspase-9.

FIG. 11, comprising FIG. 11A through FIG. 11D, is a series of images depicting inducible hNanog expression by ES cells. FIG. 11A is a schematic illustration of the hNanog gene and a mutated version carrying the point mutation D69E were subcloned. These fragments were integrated upstream of the HPRT locus in A2lox ES cells via Cre-mediated recombination, placing them under the control of a tetracycline response element (TRE). The tet transactivator (rtTA) is targeted to the Rosa26 locus on chromosome 6. FIG. 11B is an image depicting PCR products obtained from RNA extracted from two cell lines stimulated with doxycycline. Only doxycycline-treated cells carrying wild-type or mutated hNanog showed increased levels of the transgene. FIG. 11C is an image depicting simultaneous detection of endogenous Nanog and the hNanog transgene in cells stimulated with increasing concentrations of doxycycline using RT-PCR. The highest concentration of doxycycline (1 μg/ml) results in a similar expression level of the hNanog transgenic as does the endogenous Nanog. FIG. 11D is a series of images depicting phase-contrast (left column), GFP channel (middle column), and flow cytometry (right column) in the mixing experiment carried out in FIG. 4F. D69E hNanog samples showed a significant number of GFP-negative cells that grew in clusters with undifferentiated ES cell morphology, whereas most of the GFP-positive cells appear to be differentiated.

### DETAILED DESCRIPTION OF THE INVENTION

The present invention is based, in part, on the discovery that a caspase specifically cleaves the transcription factor, Nanog, leading to the initiation of cellular differentiation of embryonic stem (ES) cells.

Accordingly, the methods of the present invention include a method of maintaining the pluripotency of an ES cell. In another embodiment, the present invention provides compositions and methods to prevent the differentiation of an ES cell. In another embodiment, the present invention provides compositions and methods for inhibiting the cleavage of Nanog. In still another embodiment, the present invention provides compositions and methods for inhibiting caspase expression, activity, and/or stability.

In yet another embodiment, the present invention provides compositions and methods for the initiation of differentiation of an ES cell.

### DEFINITIONS

As used herein, each of the following terms has the meaning associated with it in this section.

The articles “a” and “an” are used herein to refer to one or to more than one (i.e. to at least one) of the grammatical object of the article. By way of example, “an element” means one element or more than one element.

The term “about” will be understood by persons of ordinary skill in the art and will vary to some extent on the context in which it is used.

As used herein “endogenous” refers to any material from or produced inside an organism, cell, tissue or system.

As used herein, the term “exogenous” refers to any material introduced from or produced outside an organism, cell, tissue or system.

The term “expression” as used herein is defined as the transcription and/or translation of a particular nucleotide sequence driven by its promoter.

The term “expression vector” as used herein refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of antisense molecules, siRNA, ribozymes, and the like. Expression vectors can contain a variety of control sequences, which refer to nucleic acid sequences necessary for the transcription and possibly translation of an operatively linked coding sequence in a particular host organism. In addition to control sequences that govern transcription and translation, vectors and expression vectors may contain nucleic acid sequences that serve other functions as well.

The phrase “inhibit,” as used herein, means to reduce a molecule, a reaction, an interaction, a gene, an mRNA, and/or a protein's expression, stability, function or activity by a measurable amount or to prevent it's expression, stability, function, or activity entirely. Inhibitors are compounds that, e.g., bind to, partially or totally block stimulation, decrease, prevent, delay activation, inactivate, desensitize, or down regulate a protein, a gene, and an mRNA stability, expression, function and activity, e.g., antagonists.

The expression “effective amount,” as used herein, refers to the amount of a compound that results in a desired effect, such as the inhibition of caspase 3's ability to cleave Nanog.

The term “stem cell,” as used herein, refers to a pluripotent or lineage-uncommitted cell which is potentially capable of an unlimited number of mitotic divisions to either renew itself or to produce progeny cells, and which is able to differentiate into multiple cell types derived from single-cell clones.

An “isolated cell” refers to a cell which has been separated from other components and/or cells which naturally accompany the cell in a tissue or mammal.

The term “pluripotent” or “pluripotency,” as used herein, refers to a cell that can differentiate into all cell types except for extra-embryonic tissue, in contrast to a “totipotent cell,” which can produce every cell type including extraembryonic tissue. A “pluripotent cell” or a “pluripotent stem cell,” as used herein, is a cell that has the potential to differentiate into any of the three germ layers: endoderm, mesoderm, or ectoderm. Accordingly, a “pluripotent cell” or “a pluripotent stem cell” can give rise to any fetal or adult cell type.

The term “maintaining the pluripotency of a cell” or “maintaining the pluripotency of an embryonic stem cell,” as used herein refers to any means, process, or method whereby the potential of a pluripotent cell to differentiate into any of the three germ layers (endoderm, mesoderm, or ectoderm) is preserved, maintained, prolonged, or extended. Alternatively, the terms “maintaining the pluripotency of a cell” or “maintaining the pluripotency of an embryonic stem cell,” as used herein, may also refer to any means, process, or method whereby the differentiation of a pluripotent cell is inhibited.

An “isolated cell” refers to a cell which has been separated from other components and/or cells which naturally accompany the cell in a tissue or mammal.

As used herein, a “substantially purified cell” is a cell that has been purified from other cell types with which it is normally associated in its naturally-occurring state.

“Expandability” is used herein to refer to the capacity of a cell to proliferate, for example, to expand in number or, in the case of a population of cells, to undergo population doublings. Cells are expanded in culture when they are placed in a growth medium under conditions that facilitate cell growth and/or division, resulting in a larger population of the cells.

As used herein, the term “growth medium” is meant to refer to a culture medium that promotes proliferation of cells.

“Proliferation” is used herein to refer to the reproduction or multiplication of similar forms, especially of cells. That is, proliferation encompasses production of a greater number of cells, and can be measured by, among other things, simply counting the numbers of cells, and the like. The rate of cell proliferation is typically measured by the amount of time required for the cells to double in number, otherwise known as the doubling time.

As used herein, “cell culture” refers to the process whereby cells, taken from a living organism, are grown under controlled conditions.

A “primary cell culture” refers to a culture of cells, tissues or organs taken directly from an organism.

As used herein, “subculture” refers to the transfer of cells from one growth container to another growth container.

“Exogenous” refers to any material introduced into or produced outside an organism, cell, or system.

As used herein, the term “phenotype” or “phenotypic characteristics” should be construed to mean the expression of a specific biomarker protein or nucleic acid, or a combination of biomarker proteins or nucleic acids, that distinguishes one cell from another.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

An “isolated nucleic acid” refers to a nucleic acid segment or fragment which has been separated from sequences which flank it in a naturally occurring state, i.e., a DNA fragment which has been removed from the sequences which are normally adjacent to the fragment, i.e., the sequences adjacent to the fragment in a genome in which it naturally occurs. The term also applies to nucleic acids which have been substantially purified from other components which naturally accompany the nucleic acid, i.e., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (i.e., as a cDNA or a genomic or cDNA fragment produced by PCR or restriction enzyme digestion) independent of other sequences. It also includes a recombinant DNA which is part of a hybrid gene encoding additional polypeptide sequence.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

“Recombinant polynucleotide” refers to a polynucleotide having sequences that are not naturally joined together. An amplified or assembled recombinant polynucleotide may be included in a suitable vector, and the vector can be used to transform a suitable host cell. A recombinant polynucleotide may serve a non-coding function (e.g., promoter, origin of replication, ribosome-binding site, etc.) as well.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

As used herein, the term “promoter/regulatory sequence” means a nucleic acid sequence which is required for expression of a gene product operably linked to the promoter/regulator sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a n inducible manner.

An “inducible” promoter is a nucleotide sequence which, when operably linked with a polynucleotide which encodes or specifies a gene product, causes the gene product to be produced substantially only when an inducer which corresponds to the promoter is present.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof. Synthetic polypeptides can be synthesized, for example, using an automated polypeptide synthesizer.

The term “protein” typically refers to large polypeptides.

The term “peptide” typically refers to short polypeptides.

By “pharmaceutically acceptable carrier” is meant any carrier, diluent or excipient which is compatible with the biological component of a pharmaceutical composition and not deleterious to the recipient.

### DESCRIPTION

In the present invention, it has been discovered that a caspase cleaves a key transcriptional factor, Nanog, responsible for maintaining the pluripotent state of an embryonic stem (ES) cell. Thus, caspase initiates the differentiation of an ES cell. Accordingly, the present invention provides a method of maintaining the pluripotency of an ES cell by preventing, inhibiting, or blocking the cleavage of Nanog in the ES cell.

In one embodiment, the present invention provides a method of maintaining the pluripotency of an ES cells by inhibiting caspase activity, expression, or stability in an ES cell by contacting the ES cell with an effective amount of a caspase inhibitor and thereby preventing the cleavage of Nanog. In one embodiment of the present invention, the caspase is caspase-3. In another embodiment of the present invention, the caspase is caspase-9.

In another embodiment, the present invention provides a method of preventing the differentiation of an ES cell by contacting the ES cell with an effective amount of a caspase inhibitor and thereby inhibiting Nanog cleavage. In still another embodiment, the present invention provides a method of inhibiting caspase activity, expression, or stability in an ES cell. In one embodiment of the present invention, the caspase is caspase-3. In another embodiment of the present invention, the caspase is caspase-9.

In still another embodiment, the present invention provides a method of maintaining the pluripotency of an ES cell by supplying a sufficient amount of a caspase-cleavage resistant form of Nanog to an ES cell or expressing cleavage-resistant Nanog in a modified ES cell.

In yet another embodiment of the invention, it may be desirable to induce differentiation of an ES cell in a controlled manner by enhancing the expression, activity, or stability of a caspase in an ES cell. In one embodiment of the present invention, the caspase is caspase-3. In another embodiment of the present invention, the caspase is caspase-9.

Inhibiting caspase activity can be accomplished using any method known to the skilled artisan. Examples of methods to inhibit caspase activity include, but are not limited to decreasing expression of an endogenous caspase gene, decreasing expression of caspase mRNA, and inhibiting activity of caspase protein. A caspase inhibitor may therefore be a compound or composition that decreases expression of a caspase gene, a compound or composition that decreases caspase mRNA half-life, stability and/or expression, or a compound or composition that inhibits caspase-3 protein function. A caspase inhibitor may be any type of compound, including but not limited to, a polypeptide, a nucleic acid, an aptamer, a peptidometic, and a small molecule, or combinations thereof.

Caspase inhibition may be accomplished either directly or indirectly. For example, caspase may be directly inhibited by compounds or compositions that directly interact with caspase protein, such as antibodies. Alternatively, caspase may be inhibited indirectly by compounds or compositions that inhibit caspase downstream effectors, or upstream regulators which up-regulate caspase expression.

Decreasing expression of an endogenous caspase gene includes providing a specific inhibitor of caspase gene expression. Decreasing expression of caspase mRNA or caspase protein includes decreasing the half-life or stability of caspase mRNA or decreasing expression of caspase mRNA. Methods of decreasing expression of caspase include, but are not limited to, methods that use an siRNA, a microRNA, an antibody, an antisense nucleic acid, a ribozyme, an expression vector encoding a transdominant negative mutant, a peptide, a small molecule, other specific inhibitors of caspase-3 gene, mRNA, and protein expression, and combinations thereof.

### Antibodies

In one embodiment of the invention, the caspase inhibitor is an antibody. It will be appreciated by one skilled in the art that an antibody comprises any immunoglobulin molecule, whether derived from natural sources or from recombinant sources, which is able to specifically bind to an epitope present on a target molecule. In the present invention, the target molecule may be caspase, or fragments thereof. In one aspect of the invention, caspase is directly inhibited by an antibody that specifically binds to an epitope on caspase. In another aspect of the invention, caspase is indirectly inhibited by an antibody.

When the caspase inhibitor used in the compositions and methods of the invention is a polyclonal antibody (IgG), the antibody is generated by inoculating a suitable animal with a peptide comprising full length caspase or fragments thereof. These polypeptides, or fragments thereof, may be obtained by any method known in the art, including chemical synthesis and biological synthesis, as described elsewhere herein. Antibodies produced in the inoculated animal which specifically bind to caspase or fragments thereof, are then isolated from fluid obtained from the animal. Antibodies may be generated in this manner in several non-human mammals such as, but not limited to goat, sheep, horse, camel, rabbit, and donkey. Methods for generating polyclonal antibodies are well known in the art and are described, for example in Harlow, et al. (1988, In: Antibodies, A Laboratory Manual, Cold Spring Harbor, N.Y.).

Monoclonal antibodies directed against a full length caspase or fragment thereof, may be prepared using any well known monoclonal antibody preparation procedures, such as those described, for example, in Harlow et al. (1998, In: Antibodies, A Laboratory Manual, Cold Spring Harbor, N.Y.) and in Tuszynski et al. (1988, Blood, 72:109-115). Human monoclonal antibodies may be prepared by the method described in U.S. patent publication 2003/0224490. Monoclonal antibodies directed against an antigen are generated from mice immunized with the antigen using standard procedures as referenced herein. Nucleic acid encoding the monoclonal antibody obtained using the procedures described herein may be cloned and sequenced using technology which is available in the art, and is described, for example, in Wright et al. (1992, Critical Rev. in Immunol. 12(3, 4):125-168) and the references cited therein.

When the antibody used in the methods of the invention is a biologically active antibody fragment or a synthetic antibody corresponding to antibody to a full length caspase or fragments thereof, the antibody is prepared as follows: a nucleic acid encoding the desired antibody or fragment thereof is cloned into a suitable vector. The vector is transfected into cells suitable for the generation of large quantities of the antibody or fragment thereof. DNA encoding the desired antibody is then expressed in the cell thereby producing the antibody. The nucleic acid encoding the desired peptide may be cloned and sequenced using technology which is available in the art, and described, for example, in Wright et al. (1992, Critical Rev. in Immunol. 12(3, 4):125-168) and the references cited therein. Alternatively, quantities of the desired antibody or fragment thereof may also be synthesized using chemical synthesis technology. If the amino acid sequence of the antibody is known, the desired antibody can be chemically synthesized using methods known in the art as described elsewhere herein.

The present invention also includes the use of humanized antibodies specifically reactive with an epitope present on a target molecule. These antibodies are capable of binding to the target molecule. The humanized antibodies useful in the invention have a human framework and have one or more complementarity determining regions (CDRs) from an antibody, typically a mouse antibody, specifically reactive with a targeted cell surface molecule.

When the antibody used in the invention is humanized, the antibody can be generated as described in Queen, et al. (U.S. Pat. No. 6,180,370), Wright et al., (supra) and in the references cited therein, or in Gu et al. (1997, Thrombosis and Hematocyst 77(4):755-759), or using other methods of generating a humanized antibody known in the art. The method disclosed in Queen et al. is directed in part toward designing humanized immunoglobulins that are produced by expressing recombinant DNA segments encoding the heavy and light chain complementarity determining regions (CDRs) from a donor immunoglobulin capable of binding to a desired antigen, attached to DNA segments encoding acceptor human framework regions. Generally speaking, the invention in the Queen patent has applicability toward the design of substantially any humanized immunoglobulin. Queen explains that the DNA segments will typically include an expression control DNA sequence operably linked to humanized immunoglobulin coding sequences, including naturally-associated or heterologous promoter regions. The expression control sequences can be eukaryotic promoter systems in vectors capable of transforming or transfecting eukaryotic host cells, or the expression control sequences can be prokaryotic promoter systems in vectors capable of transforming or transfecting prokaryotic host cells. Once the vector has been incorporated into the appropriate host, the host is maintained under conditions suitable for high level expression of the introduced nucleotide sequences and as desired the collection and purification of the humanized light chains, heavy chains, light/heavy chain dimers or intact antibodies, binding fragments or other immunoglobulin forms may follow (Beychok, Cells of Immunoglobulin Synthesis, Academic Press, New York, (1979), which is incorporated herein by reference).

Human constant region (CDR) DNA sequences from a variety of human cells can be isolated in accordance with well known procedures. Preferably, the human constant region DNA sequences are isolated from immortalized B-cells as described in WO 87/02671. CDRs useful in producing the antibodies of the present invention may be similarly derived from DNA encoding monoclonal antibodies capable of binding to the target molecule. Such humanized antibodies may be generated using well known methods in any convenient mammalian source capable of producing antibodies, including, but not limited to, mice, rats, camels, llamas, rabbits, or other vertebrates. Suitable cells for constant region and framework DNA sequences and host cells in which the antibodies are expressed and secreted, can be obtained from a number of sources, such as the American Type Culture Collection, Manassas, Va.

One of skill in the art will further appreciate that the present invention encompasses the use of antibodies derived from camelid species. That is, the present invention includes, but is not limited to, the use of antibodies derived from species of the camelid family. As is well known in the art, camelid antibodies differ from those of most other mammals in that they lack a light chain, and thus comprise only heavy chains with complete and diverse antigen binding capabilities (Hamers-Casterman et al., 1993, Nature, 363:446-448). Such heavy-chain antibodies are useful in that they are smaller than conventional mammalian antibodies, they are more soluble than conventional antibodies, and further demonstrate an increased stability compared to some other antibodies. Camelid species include, but are not limited to Old World camelids, such as two-humped camels (C. bactrianus) and one humped camels (C. dromedarius). The camelid family further comprises New World camelids including, but not limited to llamas, alpacas, vicuna and guanaco. The production of polyclonal sera from camelid species is substantively similar to the production of polyclonal sera from other animals such as sheep, donkeys, goats, horses, mice, chickens, rats, and the like. The skilled artisan, when equipped with the present disclosure and the methods detailed herein, can prepare high-titers of antibodies from a camelid species. As an example, the production of antibodies in mammals is detailed in such references as Harlow et al., (1988, Antibodies: A Laboratory Manual, Cold Spring Harbor, N.Y.).

V_{H} proteins isolated from other sources, such as animals with heavy chain disease (Seligmann et al., 1979, Immunological Rev. 48:145-167, incorporated herein by reference in its entirety), are also useful in the compositions and methods of the invention. The present invention further comprises variable heavy chain immunoglobulins produced from mice and other mammals, as detailed in Ward et al. (1989, Nature 341:544-546, incorporated herein by reference in its entirety). Briefly, V_{H} genes are isolated from mouse splenic preparations and expressed in E. coli. The present invention encompasses the use of such heavy chain immunoglobulins in the compositions and methods detailed herein.

Antibodies useful as caspase inhibitors in the invention may also be obtained from phage antibody libraries. To generate a phage antibody library, a cDNA library is first obtained from mRNA which is isolated from cells, e.g., the hybridoma, which express the desired protein to be expressed on the phage surface, e.g., the desired antibody. cDNA copies of the mRNA are produced using reverse transcriptase. cDNA which specifies immunoglobulin fragments are obtained by PCR and the resulting DNA is cloned into a suitable bacteriophage vector to generate a bacteriophage DNA library comprising DNA specifying immunoglobulin genes. The procedures for making a bacteriophage library comprising heterologous DNA are well known in the art and are described, for example, in Sambrook et al. (2001, Molecular Cloning: A Laboratory Manual, Cold Spring Harbor Laboratory Press, Cold Spring Harbor, N.Y.).

Bacteriophage which encode the desired antibody, may be engineered such that the protein is displayed on the surface thereof in such a manner that it is available for binding to its corresponding binding protein, e.g., the antigen against which the antibody is directed. Thus, when bacteriophage which express a specific antibody are incubated in the presence of a cell which expresses the corresponding antigen, the bacteriophage will bind to the cell. Bacteriophage which do not express the antibody will not bind to the cell. Such panning techniques are well known in the art and are described for example, in Wright et al., (supra).

Processes such as those described above, have been developed for the production of human antibodies using M13 bacteriophage display (Burton et al., 1994, Adv. Immunol. 57:191-280). Essentially, a cDNA library is generated from mRNA obtained from a population of antibody-producing cells. The mRNA encodes rearranged immunoglobulin genes and thus, the cDNA encodes the same. Amplified cDNA is cloned into M13 expression vectors creating a library of phage which express human Fab fragments on their surface. Phage which display the antibody of interest are selected by antigen binding and are propagated in bacteria to produce soluble human Fab immunoglobulin. Thus, in contrast to conventional monoclonal antibody synthesis, this procedure immortalizes DNA encoding human immunoglobulin rather than cells which express human immunoglobulin.

The procedures just presented describe the generation of phage which encode the Fab portion of an antibody molecule. However, the invention should not be construed to be limited solely to the generation of phage encoding Fab antibodies. Rather, phage which encode single chain antibodies (scFv/phage antibody libraries) are also included in the invention. Fab molecules comprise the entire Ig light chain, that is, they comprise both the variable and constant region of the light chain, but include only the variable region and first constant region domain (CHI) of the heavy chain. Single chain antibody molecules comprise a single chain of protein comprising the Ig Fv fragment. An Ig Fv fragment includes only the variable regions of the heavy and light chains of the antibody, having no constant region contained therein. Phage libraries comprising scFv DNA may be generated following the procedures described in Marks et al., 1991, J. Mol. Biol. 222:581-597. Panning of phage so generated for the isolation of a desired antibody is conducted in a manner similar to that described for phage libraries comprising Fab DNA.

The invention should also be construed to include synthetic phage display libraries in which the heavy and light chain variable regions may be synthesized such that they include nearly all possible specificities (Barbas, 1995, Nature Medicine 1:837-839; de Kruif et al., 1995, J. Mol. Biol. 248:97-105).

Once expressed, whole antibodies, dimers derived therefrom, individual light and heavy chains, or other forms of antibodies can be purified according to standard procedures known in the art. Such procedures include, but are not limited to, ammonium sulfate precipitation, the use of affinity columns, routine column chromatography, gel electrophoresis, and the like (see, generally, R. Scopes, “Protein Purification”, Springer-Verlag, N.Y. (1982)). Substantially pure antibodies of at least about 90% to 95% homogeneity are preferred, and antibodies having 98% to 99% or more homogeneity most preferred for pharmaceutical uses. Once purified, the antibodies may then be used to practice the method of the invention, or to prepare a pharmaceutical composition useful in practicing the method of the invention.

The antibodies of the present invention can be assayed for immunospecific binding by any method known in the art. The immunoassays which can be used include but are not limited to competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few. Such assays are routine and well known in the art (see, e.g, Current Protocols in Molecular Biology, (Ausubel et al., eds.), Greene Publishing Associates and Wiley-Interscience, New York (2002)).

siRNA

In one embodiment, siRNA is used to decrease the level of caspase protein. RNA interference (RNAi) is a phenomenon in which the introduction of double-stranded RNA (dsRNA) into a diverse range of organisms and cell types causes degradation of the complementary mRNA. In the cell, long dsRNAs are cleaved into short 21-25 nucleotide small interfering RNAs, or siRNAs, by a ribonuclease known as Dicer. The siRNAs subsequently assemble with protein components into an RNA-induced silencing complex (RISC), unwinding in the process. Activated RISC then binds to complementary transcript by base pairing interactions between the siRNA antisense strand and the mRNA. The bound mRNA is cleaved and sequence specific degradation of mRNA results in gene silencing. See, for example, U.S. Pat. No. 6,506,559; Fire et al., 1998, Nature 391(19):306-311; Timmons et al., 1998, Nature 395:854; Montgomery et al., 1998, TIG 14 (7):255-258; David R. Engelke, Ed., RNA Interference (RNAi) Nuts & Bolts of RNAi Technology, DNA Press, Eagleville, Pa. (2003); and Gregory 3. Hannon, Ed., RNAi A Guide to Gene Silencing, Cold Spring Harbor Laboratory Press, Cold Spring Harbor, N.Y. (2003). Soutschek et al. (2004, Nature 432:173-178) describe a chemical modification to siRNAs that aids in intravenous systemic delivery. Optimizing siRNAs involves consideration of overall G/C content, C/T content at the termini, Tm and the nucleotide content of the 3′ overhang. See, for instance, Schwartz et al., 2003, Cell, 115:199-208 and Khvorova et al., 2003, Cell 115:209-216. Therefore, the present invention also includes methods of decreasing levels of caspase 3 protein using RNAi technology.

### Antisense Nucleic Acids

In one embodiment of the invention, an antisense nucleic acid sequence which is expressed by a plasmid vector is used to inhibit caspase. The antisense expressing vector is used to transfect a mammalian cell or the mammal itself, thereby causing reduced endogenous expression of caspase.

Antisense molecules and their use for inhibiting gene expression are well known in the art (see, e.g., Cohen, 1989, In: Oligodeoxyribonucleotides, Antisense Inhibitors of Gene Expression, CRC Press). Antisense nucleic acids are DNA or RNA molecules that are complementary, as that term is defined elsewhere herein, to at least a portion of a specific mRNA molecule (Weintraub, 1990, Scientific American 262:40). In the cell, antisense nucleic acids hybridize to the corresponding mRNA, forming a double-stranded molecule thereby inhibiting the translation of genes.

The use of antisense methods to inhibit the translation of genes is known in the art, and is described, for example, in Marcus-Sakura (1988, Anal. Biochem. 172:289). Such antisense molecules may be provided to the cell via genetic expression using DNA encoding the antisense molecule as taught by Inoue, 1993, U.S. Pat. No. 5,190,931.

Alternatively, antisense molecules of the invention may be made synthetically and then provided to the cell. Antisense oligomers of between about 10 to about 30, and more preferably about 15 nucleotides, are preferred, since they are easily synthesized and introduced into a target cell. Synthetic antisense molecules contemplated by the invention include oligonucleotide derivatives known in the art which have improved biological activity compared to unmodified oligonucleotides (see U.S. Pat. No. 5,023,243).

### Ribozymes

Ribozymes and their use for inhibiting gene expression are also well known in the art (see, e.g., Cech et al., 1992, J. Biol. Chem. 267:17479-17482; Hampel et al., 1989, Biochemistry 28:4929-4933; Eckstein et al., International Publication No. WO 92/07065; Altman et al., U.S. Pat. No. 5,168,053). Ribozymes are RNA molecules possessing the ability to specifically cleave other single-stranded RNA in a manner analogous to DNA restriction endonucleases. Through the modification of nucleotide sequences encoding these RNAs, molecules can be engineered to recognize specific nucleotide sequences in an RNA molecule and cleave it (Cech, 1988, J. Amer. Med. Assn. 260:3030). A major advantage of this approach is the fact that ribozymes are sequence-specific.

There are two basic types of ribozymes, namely, tetrahymena-type (Hasselhoff, 1988, Nature 334:585) and hammerhead-type. Tetrahymena-type ribozymes recognize sequences which are four bases in length, while hammerhead-type ribozymes recognize base sequences 11-18 bases in length. The longer the sequence, the greater the likelihood that the sequence will occur exclusively in the target mRNA species. Consequently, hammerhead-type ribozymes are preferable to tetrahymena-type ribozymes for inactivating specific mRNA species, and 18-base recognition sequences are preferable to shorter recognition sequences which may occur randomly within various unrelated mRNA molecules.

In one embodiment of the invention, a ribozyme is used to inhibit caspase expression. Ribozymes useful for inhibiting the expression of a target molecule may be designed by incorporating target sequences into the basic ribozyme structure which are complementary, for example, to the mRNA sequence of caspase of the present invention. Ribozymes targeting caspase may be synthesized using commercially available reagents (Applied Biosystems, Inc., Foster City, Calif.) or they may be genetically expressed from DNA encoding them.

### Peptides

Caspase inhibitors that are peptides and cleavage-resistant Nanog peptides useful in the invention may be obtained using standard methods known to the skilled artisan. Such methods include chemical organic synthesis or biological means. Biological means include purification from a biological source, recombinant synthesis and in vitro translation systems, using methods well known in the art.

A peptide may be chemically synthesized by Merrifield-type solid phase peptide synthesis. This method may be routinely performed to yield peptides up to about 60-70 residues in length, and may, in some cases, be utilized to make peptides up to about 100 amino acids long. Larger peptides may also be generated synthetically via fragment condensation or native chemical ligation (Dawson et al., 2000, Ann. Rev. Biochem. 69:923-960). An advantage to the utilization of a synthetic peptide route is the ability to produce large amounts of peptides, even those that rarely occur naturally, with relatively high purities, i.e., purities sufficient for research, diagnostic or therapeutic purposes.

Solid phase peptide synthesis is described by Stewart et al. in Solid Phase Peptide Synthesis, 2nd Edition, 1984, Pierce Chemical Company, Rockford, Ill.; and Bodanszky and Bodanszky in The Practice of Peptide Synthesis, 1984, Springer-Verlag, New York. At the outset, a suitably protected amino acid residue is attached through its carboxyl group to a derivatized, insoluble polymeric support, such as cross-linked polystyrene or polyamide resin. “Suitably protected” refers to the presence of protecting groups on both the α-amino group of the amino acid, and on any side chain functional groups. Side chain protecting groups are generally stable to the solvents, reagents and reaction conditions used throughout the synthesis, and are removable under conditions which will not affect the final peptide product. Stepwise synthesis of the oligopeptide is carried out by the removal of the N-protecting group from the initial amino acid, and coupling thereto of the carboxyl end of the next amino acid in the sequence of the desired peptide. This amino acid is also suitably protected. The carboxyl of the incoming amino acid can be activated to react with the N-terminus of the support-bound amino acid by formation into a reactive group, such as formation into a carbodiimide, a symmetric acid anhydride, or an “active ester” group, such as hydroxybenzotriazole or pentafluorophenyl esters.

Examples of solid phase peptide synthesis methods include the BOC method, which utilizes tert-butyloxcarbonyl as the α-amino protecting group, and the FMOC method, which utilizes 9-fluorenylmethyloxcarbonyl to protect the α-amino of the amino acid residues. Both methods are well-known by those of skill in the art.

Incorporation of N- and/or C-blocking groups may also be achieved using protocols conventional to solid phase peptide synthesis methods. For incorporation of C-terminal blocking groups, for example, synthesis of the desired peptide is typically performed using, as solid phase, a supporting resin that has been chemically modified so that cleavage from the resin results in a peptide having the desired C-terminal blocking group. To provide peptides in which the C-terminus bears a primary amino blocking group, for instance, synthesis is performed using a p-methylbenzhydrylamine (MBHA) resin, so that, when peptide synthesis is completed, treatment with hydrofluoric acid releases the desired C-terminally amidated peptide. Similarly, incorporation of an N-methylamine blocking group at the C-terminus is achieved using N-methylaminoethyl-derivatized DVB (divinylbenzene), resin, which upon hydrofluoric acid (HF) treatment releases a peptide bearing an N-methylamidated C-terminus. Blockage of the C-terminus by esterification can also be achieved using conventional procedures. This entails use of resin/blocking group combination that permits release of side-chain peptide from the resin, to allow for subsequent reaction with the desired alcohol, to form the ester function. FMOC protecting group, in combination with DVB resin derivatized with methoxyalkoxybenzyl alcohol or equivalent linker, can be used for this purpose, with cleavage from the support being effected by trifluoroacetic acid (TFA) in dicholoromethane. Esterification of the suitably activated carboxyl function, e.g. with dicyclohexylcarbodiimide (DCC), can then proceed by addition of the desired alcohol, followed by de-protection and isolation of the esterified peptide product.

Incorporation of N-terminal blocking groups may be achieved while the synthesized peptide is still attached to the resin, for instance by treatment with a suitable anhydride and nitrile. To incorporate an acetyl blocking group at the N-terminus, for instance, the resin-coupled peptide can be treated with 20% acetic anhydride in acetonitrile. The N-blocked peptide product may then be cleaved from the resin, de-protected and subsequently isolated.

Prior to its use as a caspase inhibitor in accordance with the invention, a peptide is purified to remove contaminants. In this regard, it will be appreciated that the peptide will be purified so as to meet the standards set out by the appropriate regulatory agencies. Any one of a number of a conventional purification procedures may be used to attain the required level of purity including, for example, reversed-phase high-pressure liquid chromatography (HPLC) using an alkylated silica column such as C₄-, C₈- or C₁₈-silica. A gradient mobile phase of increasing organic content is generally used to achieve purification, for example, acetonitrile in an aqueous buffer, usually containing a small amount of trifluoroacetic acid. Ion-exchange chromatography can be also used to separate polypeptides based on their charge. Affinity chromatography is also useful in purification procedures.

Antibodies and peptides may be modified using ordinary molecular biological techniques to improve their resistance to proteolytic degradation or to optimize solubility properties or to render them more suitable as a therapeutic agent. Analogs of such polypeptides include those containing residues other than naturally occurring L-amino acids, e.g., D-amino acids or non-naturally occurring synthetic amino acids. The polypeptides useful in the invention may further be conjugated to non-amino acid moieties that are useful in their application. In particular, moieties that improve the stability, biological half-life, water solubility, and immunologic characteristics of the peptide are useful. A non-limiting example of such a moiety is polyethylene glycol (PEG).

### Embryonic Stem Cells

The present invention encompasses any embryonic stem cell or embryonic stem cell line, both known and unknown, in the art. Methods for procuring, culturing, an maintaining an embryonic stem (ES) cell or an ES cell line of the present invention are well known in the art. Indeed, ES cells from various mammalian embryos have been successfully grown in the laboratory. Evans and Kaufman, 1981, (Nature 292:154-156) and Martin, 1981, (PNAS 72:1441-1445) showed that it is possible to derive permanent lines of embryonic cells directly from mouse blastocysts. Thomson successfully derived permanent cell lines from rhesus and marmoset monkeys (Thomson et al., 1996, Biol. Reprod. 55:254-259; Thomson et al., 1995, PNAS 92:7844-7848). Pluripotent cell lines have also been derived from pre-implantation embryos of several domestic and laboratory animal species such as bovines (Evans et al., 1990, Theriogenology 33:125-128) Porcine (Evans et al., 1990, Theriogenology 33:125-128; Notarianni et al., 1990 J. Reprod. Fertil. Suppl. 41:51-56), Sheep and goat (Meinecke-Tillmann and Meinecke, 1996, J. Animal Breeding and Genetics 113:413-426; Notarianni et al., 1991, J. Reprod. Fertil. Suppl. 43:255-260), rabbit (Giles et al., 1993, Mol. Reprod. Dev. 36:130-138; Graves et al., 1993, Mol. Reprod. Dev. 36:424-433) Mink (Sukoyan et al., 1992, Mol. Reprod. Dev. 33:418-431)) rat (Iannaccona et al., 1994, Dev. Biol. 163:288-292) and Hamster (Doetschman et al., 1988, Dev. Biol. 127:224-227). Recently, Thomson et al., 1998, (Science 282:1145-1147) and Reubinoff et al., 2000, (Nature Biotech. 18:299-304) have reported the derivation of human ES cell lines. These human ES cells resemble the rhesus monkey ES cell lines.

ES cells are isolated from the ICM of the blastocyst, an early stage of the developing embryo. The blastocyst is the stage of embryonic development prior to implantation that contains two types of cells: trophectoderm, outer layer which gives rise to extra embryonic membranes, and the inner cell mass (ICM) which forms the embryo proper.

ES cells isolated from the ICM during the blastocyst stage, however, can be cultured in the laboratory and under the right conditions proliferate indefinitely. ES cells growing in this undifferentiated state retain the potential to differentiate into cells of all three embryonic tissue layers. Ultimately, the cells of the inner cell mass give rise to all the embryonic tissues. It is at this stage of embryogenesis, that ES cells can be derived from the ICM of the blastocyst.

The ability to isolate ES cells from blastocysts and grow them in culture seems to depend in large part on the integrity and condition of the blastocyst from which the cells are derived. In short, the blastocyst that is large and has distinct inner cell mass tends to yield ES cells most efficiently. Several methods have been used for isolation of inner cell mass (ICM) for the establishment of embryonic stem cell lines. The most common methods are as follows:

Natural Hatching of the Blastocyst In this procedure blastocyst is allowed to hatch naturally after plating on the feeder layer. The inner cell mass (ICM) of the hatched blastocyst develops an outgrowth. This outgrowth is removed mechanically and is subsequently grown for establishing embryonic stem cell lines.

Microsurgery Another method of isolation of inner cell mass is mechanical aspiration called microsurgery. In this process, the blastocyst is held by the holding pipette using micromanipulator system and positioned in such a way that the inner cell mass (ICM) is at 9 o'clock position. The inner cell mass (ICM) is aspirated using a bevel-shaped biopsy needle which is inserted into the blastocoel cavity. The operation at the cellular level requires tools with micrometer precision, thereby minimizing damage and contamination.

Immunosurgery Immunosurgery is a commonly used procedure to isolate inner cell mass (ICM). The inner cell mass (ICM) is isolated by complement mediated lysis. In this procedure, the blastocyst is exposed either to acid tyrode solution or pronase enzyme solution in order to remove the zona pellucida (shell) of blastocyst. The zona free embryo is then exposed to human surface antibody for about 30 min to one hour. This is followed by exposure of embryos to guinea pig complement in order to lyse the trophectoderm. The complement mediated lysed trophectoderm cells are removed from inner cell mass (ICM) by repeated mechanical pipetting with a finely drawn Pasteur pipette. All the embryonic stem cell lines reported currently in the literature have been derived by this method. However, this method has several disadvantages. First the embryo is exposed for a long time to acid tyrode or pronase causing deleterious effects on embryo, thereby reducing the viability of embryos. Second, it is a time consuming procedure as it takes about 1.5 to 2.0 hours. (Narula et al., 1996, Mol. Reprod. Dev. 44:343-351). Third, the yield of inner cell mass (ICM) per blastocyst is low. Fourth, critical storage conditions are required for antibody and complement used in the process. Last, it involves the risk of transmission of virus and bacteria of animal origin to humans, as animal derived antibodies and complement are used in the process. In this process, two animal sera are used. One is rabbit antihuman antiserum and the other is guinea pig complement sera. The human cell lines studied to date are mainly derived by using a method of immunosurgery, where animal based antisera and complement was used.

### Modified ES Cells

In one embodiment, the present invention contemplates ES cells expressing a caspase-cleavage resistant form of Nanog, as taught herein. Accordingly, the invention further contemplates the ability to transduce embryonic stem cells and/or their differentiated progeny with particular nucleic acids, thereby giving rise to genetically modified stem cells and progeny. Transduction of human embryonic stem cell (hESC) lines is also taught in US Patent Application Publication No. 20050079616.

As used herein, “transduction of embryonic stem cells” refers to the process of transferring exogenous genetic material into an embryonic stem cell. The terms “transduction”, “transfection” and “transformation” are used interchangeably herein, and refer to the process of transferring exogenous genetic material into a cell. As used herein, “exogenous genetic material” refers to nucleic acids or oligonucleotides, either natural or synthetic, that are introduced into the cells. The exogenous genetic material may be a copy of that which is naturally present in the cells, or it may not be naturally found in the cells. It typically is at least a portion of a naturally occurring gene which has been placed under operable control of a promoter in a vector construct.

Various techniques may be employed for introducing nucleic acids into cells. Such techniques include electroporation, transfection of nucleic acid-CaPO₄ precipitates, transfection of nucleic acids associated with DEAE, transfection with a retrovirus including the nucleic acid of interest, liposome mediated transfection, and the like. For certain uses, it is preferred to target the nucleic acid to particular cells. In such instances, a vehicle used for delivering a nucleic acid according to the invention into a cell (e.g., a retrovirus, or other virus; a liposome) can have a targeting molecule attached thereto. For example, a molecule such as an antibody specific for a surface membrane protein on the target cell or a ligand for a receptor on the target cell can be bound to or incorporated within the nucleic acid delivery vehicle. For example, where liposomes are employed to deliver the nucleic acids of the invention, proteins which bind to a surface membrane protein associated with endocytosis may be incorporated into the liposome formulation for targeting and/or to facilitate uptake. Such proteins include proteins or fragments thereof tropic for a particular cell type, antibodies for proteins which undergo internalization, proteins that confer intracellular localization and/or enhance intracellular half life, and the like. Polymeric delivery systems also have been used successfully to deliver nucleic acids into cells, as is known by those skilled in the art.

One method of introducing exogenous genetic material into cells is through the use of replication-deficient retroviruses. Replication-deficient retroviruses are capable of directing synthesis of all virion proteins, but are incapable of making infectious particles. Accordingly, these genetically altered retroviral vectors have general utility for high-efficiency transduction of genes in cultured cells, and specific utility for use in the method of the present invention. Retroviruses have been used extensively for transferring genetic material into cells. Standard protocols for producing replication-deficient retroviruses (including the steps of incorporation of exogenous genetic material into a plasmid, transfection of a packaging cell line with plasmid, production of recombinant retroviruses by the packaging cell line, collection of viral particles from tissue culture media, and infection of the target cells with the viral particles) are provided in the art. A preferred retroviral expression vector includes an exogenous promoter element to control transcription of the inserted exogenous gene. Such exogenous promoters include both constitutive and inducible promoters.

The major advantage of using retroviruses is that the viruses insert efficiently a single copy of the gene encoding the therapeutic agent into the host cell genome, thereby permitting the exogenous genetic material to be passed on to the progeny of the cell when it divides. In addition, gene promoter sequences in the LTR region have been reported to enhance expression of an inserted coding sequence in a variety of cell types. The major disadvantages of using a retrovirus expression vector are (1) insertional mutagenesis, i.e., the insertion of the therapeutic gene into an undesirable position in the target cell genome which, for example, leads to unregulated cell growth and (2) the need for target cell proliferation in order for the therapeutic gene carried by the vector to be integrated into the target genome. Despite these apparent limitations, delivery of a therapeutically effective amount of a therapeutic agent via a retrovirus can be efficacious if the efficiency of transduction is high and/or the number of target cells available for transduction is high.

Yet another viral candidate useful as an expression vector for transformation of cells is the adenovirus, a double-stranded DNA virus. Like the retrovirus, the adenovirus genome is adaptable for use as an expression vector for gene transduction, i.e., by removing the genetic information that controls production of the virus itself. Because the adenovirus functions usually in an extrachromosomal fashion, the recombinant adenovirus does not have the theoretical problem of insertional mutagenesis. On the other hand, adenoviral transformation of a target cell may not result in stable transduction. However, more recently it has been reported that certain adenoviral sequences confer intrachromosomal integration specificity to carrier sequences, and thus result in a stable transduction of the exogenous genetic material.

Thus, as will be apparent to one of ordinary skill in the art, a variety of suitable vectors are available for transferring exogenous genetic material into cells. The selection of an appropriate vector to deliver a therapeutic agent for a particular condition amenable to gene replacement therapy and the optimization of the conditions for insertion of the selected expression vector into the cell, are within the scope of one of ordinary skill in the art without the need for undue experimentation. The promoter characteristically has a specific nucleotide sequence necessary to initiate transcription. Optionally, the exogenous genetic material further includes additional sequences (i.e., enhancers) required to obtain the desired gene transcription activity. For the purpose of this discussion an “enhancer” is simply any nontranslated DNA sequence which works with the coding sequence (in cis) to change the basal transcription level dictated by the promoter. Preferably, the exogenous genetic material is introduced into the genome immediately downstream from the promoter so that the promoter and coding sequence are operatively linked so as to permit transcription of the coding sequence.

Naturally-occurring constitutive promoters control the expression of essential cell functions. As a result, a gene under the control of a constitutive promoter is expressed under all conditions of cell growth. Exemplary constitutive promoters include the promoters for the following genes which encode certain constitutive or “housekeeping” functions: hypoxanthine phosphoribosyl transferase (HPRT), dihydrofolate reductase (DHFR) (Scharfmann et al., Proc. Natl. Acad. Sci. USA 88:4626-4630 (1991)), adenosine deaminase, phosphoglycerol kinase (PGK), pyruvate kinase, phosphoglycerol mutase, the actin promoter (Lai et al., Proc. Natl. Acad. Sci. USA 86: 10006-10010 (1989)), and other constitutive promoters known to those of skill in the art. In addition, many viral promoters function constitutively in eukaryotic cells. These include: the early and late promoters of SV40; the long terminal repeats (LTRS) of Moloney Leukemia Virus and other retroviruses; and the thymidine kinase promoter of Herpes Simplex Virus, among many others. Accordingly, any of the above-referenced constitutive promoters can be used to control transcription of a heterologous gene insert.

Genes that are under the control of inducible promoters are expressed only or to a greater degree, in the presence of an inducing agent, (e.g., transcription under control of the metallothionein promoter is greatly increased in presence of certain metal ions). Inducible promoters include responsive elements (REs) which stimulate transcription when their inducing factors are bound. For example, there are REs for serum factors, steroid hormones, retinoic acid and cyclic AMP. Promoters containing a particular RE can be chosen in order to obtain an inducible response and in some cases, the RE itself may be attached to a different promoter, thereby conferring inducibility to the recombinant gene. Thus, by selecting the appropriate promoter (constitutive versus inducible; strong versus weak), it is possible to control both the existence and level of expression of a therapeutic agent in the genetically modified cell. Selection and optimization of these factors for delivery of a therapeutically effective dose of a particular therapeutic agent is deemed to be within the scope of one of ordinary skill in the art without undue experimentation, taking into account the above-disclosed factors and the clinical profile of the patient.

In addition to at least one promoter and at least one heterologous nucleic acid encoding the therapeutic agent, the expression vector preferably includes a selection gene, for example, a neomycin resistance gene, for facilitating selection of cells that have been transfected or transduced with the expression vector. Alternatively, the cells are transfected with two or more expression vectors, at least one vector containing the gene(s) encoding the therapeutic agent(s), the other vector containing a selection gene. The selection of a suitable promoter, enhancer, selection gene and/or signal sequence (described below) is deemed to be within the scope of one of ordinary skill in the art without undue experimentation.

The selection and optimization of a particular expression vector for expressing a specific gene product in a cell is accomplished by obtaining the gene, preferably with one or more appropriate control regions (e.g., promoter, insertion sequence); preparing a vector construct comprising the vector into which is inserted the gene; transfecting or transducing cultured cells in vitro with the vector construct; and determining whether the gene product is present in the cells.

Suitable promoters, enhancers, vectors, etc., for such genes are published in the literature associated with the foregoing trials. In general, useful genes replace or supplement function, including genes encoding missing enzymes such as adenosine deaminase (ADA) which has been used in clinical trials to treat ADA deficiency and cofactors such as insulin and coagulation factor VIII. Genes which affect regulation can also be administered, alone or in combination with a gene supplementing or replacing a specific function. For example, a gene encoding a protein which suppresses expression of a particular protein-encoding gene can be administered.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

The materials and methods employed in the experiments disclosed herein are now described.

### ES Cell Cultures

The mouse ES cell lines used were: D3 (from ATCC, #CRL11632), R1 (from Andras Nagy, Toronto), E14Tg2a-derived A2lox (from Michael Kyba, Utah Southwestern Medical Center); cells were typically used between passages 10 and 20. A2lox ES cells express the reverse tetracycline transactivator from the endogenous Rosa26 locus and carry an insertion containing a tetracycline response element, loxP-lox2272 sites and the neomycinΔATG-resistance gene upstream of HPRT on the X chromosome. To generate inducible derivatives, we subcloned cDNAs into the p2Lox targeting vector. Cre/Lox recombination was used to insert p2Lox into the inducible locus as described previously (Kyba et al., 2003). The medium contained 10% FBS (Innovative Research, cat. no. IFBF-H) tested for toxicity to ES cells in a colony-forming assay (Evans, 2004), that included Knockout DMEM (Invitrogen, cat. no. 10829-018), 1× nonessential amino acids (Invitrogen, cat. no. 111140-050), L-glutamine 1% v/v (Invitrogen, cat. no. 25030-081), 2-mercaptoethanol (3.5 μl per 250 ml medium; Sigma, cat. no. M7522), LIF 1000 per ml (Esgro, cat. no. ESG1107). Cells were routinely maintained on irradiated (8400 rad) mouse embryonic fibroblasts (MEFs) derived from E14.5-old embryos (mouse strain CF1, Charles River timed pregnant mice). For some experiments ES cells were placed onto 0.1% gelatin type A (Sigma, cat. no. G1890)-coated dishes and passaged 2 to 4 times before the experiment was carried out. For differentiation studies, standard medium was replaced with medium containing 1 μM retinoic acid (RA, Sigma, cat. no. R2625) without LIF or N2B27 medium (Ying et al., 2003): 45% DMEM-F12 (Invitrogen, cat. no. 11320-033); 45% Neurobasal medium (Invitrogen, cat. no. 21103-049); 2% Supplement B27 (Invitrogen, cat. no. 17504-044); 50 μg/ml BSA fraction V (Invitrogen, cat. no. 15260-037); 100 μg/ml Apo-Transferrin bovine (Invitrogen, cat. no. 11108-016); 25 μg/ml Recombinant Human insulin (Invitrogen, cat. no. 0030110SA); 6 ng/ml progesterone (Sigma, cat. no. P6149); 16 μg/ml putrescin (Sigma, cat. no. P6024); 30 nM Na-selenite (Sigma, cat. no. S5261); 2 mM glutamine (Invitogen, cat. no. 25030-081); and 0.15 mM monothioglycerol (Sigma, cat. no. M6145).

### In Vitro Caspase Cleavage Assay

Trypsinized ES cells (R1; 200,000; D3 250,000) were plated into 6 wells containing normal ES cell medium, and on the next day the medium was changed to 1 μM RA differentiation medium. Cells were harvested with trypsin-EDTA (Invitrogen, cat. no. 25300-054), and protein extracts were made in 0.1% CHAPS buffer, pH 7.2 (50 mM Hepes; 150 mM KCl; 1× protease inhibitors (Roche, cat. no. 1697498001); 2 mM Na fluoride and orthovanadate, respectively, at specified time points. The CaspGlo (Promega, cat. no. G8091) kit was used to directly measure caspase activity. Before determining caspase activity, we tested the system for variability within the dynamic range, using recombinant caspase-3 (BD Bioscience, cat. no. 556471). The signal was measured with the Wallac 1429 Victorll instrument (Perkin-Elmer, Wellesley, Mass.).

### Apoptosis Rate

R1 ES cells (1×10⁵) were plated into 6-cm dishes on irradiated MEFs and stimulated with RA differentiation medium for up to 72 hours. Cells were fixed with 4% paraformaldehyde (EMS, cat. no. 15710), stained with DAPI and mounted with Vectashield (Vector Lab, cat. no. H1200). The number of apoptotic figures was determined in triplicate per 100 cells. As a positive control, ES cells were incubated with staurosporine (Sigma, cat. no. 55921, 1 μM) for 4 hours.

### Caspase Sensor

The pCasp3-sensor (Clontech) was used, and the sensor insert (950 bp) was amplified by using primers with SalI 5′ (gatgtcgactcagatccgctagccgcca; SEQ ID NO. 1) and ScaI 3′ (gagtactttatctagatccggtggatcc; SEQ ID NO. 2) ends and cloned into the SalI-SmaI cloning site of the p2Lox vector (Michael Kyba). P2LoxPcaspase3 sensor (20 μg) was co-electroporated with 20 ug of pSalk-Cre (Michael Kyba) into (1×10⁶) A2lox ES cells (960 μF, 220 voltage, 25 millisec pulses). After electroporation, the cells were plated on neomycin-resistant MEFs (Chemicon, cat. no. PMEF-NL) and selected with G418 (400 μg/ml), individual clones were then picked and expanded. For the differentiation studies, a single clone was used (clone #6), 4000 cells/well were plated onto gelatin coated 8-chamber slides (Nalgene Nunc International) and stimulated with RA differentiation medium for specified times (cells incubated with staurosporine, 1 mM for 12 hours, served as a positive control). Cells were fixed with Cytofix (BD Biosciences, cat. no. 554714) at room temperature for 30 minutes, washed 3 times with Perm/Wash™ buffer, incubated with anti-EGFP antibody (1:500, MBL), diluted in Perm/Wash™ buffer (BD Biosciences, cat. no. 554714) at 4° C. overnight, washed 3 times with Perm/Wash™ buffer 3, and incubated with anti-rabbit antibody (Alexa Fluor 594, Molecular Probes, cat. no. A11072). They were mounted with VECTASHIELD plus DAPI. (Vector Labs). Cells that contained signal in the cytoplasm or in the nucleus were counted (100 cells per condition) and representative images were taken with the DeltaVision® deconvolution microscope (Applied Precision) equipped 40× objective lens. The acquired images were analyzed with soft WoRx® Suite (Applied Precision).

### PARP-1 Cleavage

D3 ES cells (25×10⁴) were plated into gelatin-coated 6-well dishes and stimulated with RA. Cells were lysed with CHAPS buffer, and 20 μg of protein was loaded onto a PAGE gel and blotted. PARP-1 was detected with anti-PARP antibody (Santa Cruz). For PARP-1 immunostaining, R1 ES cells were plated onto 4-well chamber slides (Nunc, cat. no. 177437) and differentiation was induced with differentiation medium for 2 days. Cells were fixed for 30 minutes at RT in 4% parafomaldehyde (EMS, cat. no. 15710) in PBS and permeabilized for 5 minutes at room temperature (0.2% Triton X-100, Sigma, cat. no. T8532, in PBS). Blocking was performed with 5% goat serum for 24 hours at room temperature followed by incubation with the primary antibody anti-PARP-1-p85 (1/100 dilution, Promega, cat. no. G7341, (Pemg et al., 2000, Science 287:1500-1503). After 4 washes with 0.1% Tween 20-PBS, cells were incubated with the secondary antibody (Abcam, cat. no. 6717, goat anti-FITC 1/500 in 0.1% Twin-20-PBS.), washed three times and mounted in Vectashield plus DAPI. Representative images were taken with the Axioplan 2 Imaging System Zeiss microscope.

### Active Caspase 3 Western Blot Analysis

R1 ES cells were plated at a density of 2×10⁵ cells per 6-well plate, kept in ES cell medium overnight and then stimulated for specified time with RA. Cells were lysed in CHAPS lysis buffer. Twenty μg of protein was run on a polyacrylmamide gel and blotted onto nitrocellulose membrane (BioRad). Antibody incubation was carried out with the primary anti-active caspase-3 antibody (Cell Signaling, cat. no. 9661; 1/1000 dilution). The Rockland anti rabbit IgG antibody (dilution 1/5000 in PBS 0.1% Twin-20, IRDay800 611-132-122) served as a second antibody. Reactions were detected with the infrared Imaging System Odyssey (Li—COR protocol, doc. no. 988-07568).

### VAD Inhibition Experiment

R1 ES cells were plated at a density of 2×10⁵ cells/ml into 6-well plates containing ES cell medium. On the next day, medium was changed to RA differentiation medium with or without VAD (100 μM; Calbiochem, cat. no. 627610). Medium was changed every day and cells fixed at room temperature for 15 minutes in 4% paraformaldehyde (Polyscience, cat. no. 18814) followed by alkaline phosphatase staining according to the manufacturer's instructions (Vector Laboratories, cat. no. SK5300 Alkphos Substrate III Blue.). Representative images were taken with a 10× objective. Colonies (1×10²) were assessed for their morphology as follows: totally differentiated colonies=no AP staining, no ES cell morphology; mostly differentiated=colonies containing small areas of undifferentiated AP-positive ES cells, but otherwise differentiated cells; partially differentiated=colonies containing more than 50% undifferentiated, AP-positive cells; undifferentiated=colonies lacking any discernible signs of differentiation.

### Generation of Caspase-3 Knockout

ES cells were derived from E3.5-old mouse embryos. Briefly on day 1 caspase-3 homozygous (Kuida et al., 1996) and control C57/BL6 female mice were injected i.p. with 5 IU of PMS (Calbiochem, cat. no. 367222). Forty-six hours later, the female mice received an i.p. injection of 5 IU human chorionic gonadotropin (HCG; Calbiochem, cat. no. 230734). Stimulated females were placed into a cage with 8-week old caspase-3-homozygous stud males. Blastocysts were collected on day 3 post-coitum in the afternoon. The blastocysts were placed onto irradiated mouse embryonic feeder cells and incubated for 3 days in ES cell medium, trypsinized and re-plated. Colonies apparent on day 7 were picked, expanded and genotyped with use of the following primers: mCasp3-S: TGTCATCTCGCTCTGGTACG (SEQ ID NO. 3); mCasp3-AS: CCCTTTCTGCCTGTCTTCTG (SEQ ID NO. 4; PCR product SIZE 310 bp); Neomycin-S: AGACAATCGGCTGCTCTGAT (SEQ ID NO. 5); Neomycin-AS: ATACTTTCTCGGCAGGAGCA (SEQ ID NO. 6; PCR product size 260 bp); mOct4-S: GGCGTTCTCTTTGGAAAGGTGTTC (SEQ ID NO. 7); mOct4-AS: CTCGAACCACATCCTTCTCT (SEQ ID NO. 8; PCR product size 312 bp); Actin-S: GGCCCAGAGCAAGAGAGGTATCC (SEQ ID NO. 9), Actin-AS: ACGCACGATTTCCCTCTCAGC (SEQ ID NO. 10; PCR product size 460 bp). For PCR we used GoTaq green master mix (Promega, cat. no. M7112) with the program: 1 cycle at 95° C. for 3 minutes followed by 40 cycles with 95° C.-58° C.-72° C. for 30 seconds followed by one extension of 10 minutes at 72° C. We generated two caspase-3 knockout cell lines (34A and 34B) and two control caspase-3 heterozygote cell line (22A and 22B). All experiments were carried out with these four cell lines and yielded essentially the same results.

### Teratoma, Embryoid Body Formation and PCR for Oct4

Caspase-3 heterozygous and homozygous ES cells were injected (2×10⁶ cell in PBS) into 5 to 7 weeks old male NU/J mice (Charles River, stock no. 002019). Tumors that eventually formed were extracted and subjected to histological analysis. For embryoid body (E.B.) formation heterozygous and homozygous 2×10⁶ ES cells were plated for 4 days in 60 mm plates, trypsinized, plated onto gelatin-coated plates and incubated in differentiation medium. For Oct4 gene expression analysis, ES cells were harvested, total RNA extracted (Qiagen, RNAeasy kit; cat. no. 74104) and reverse transcriptase reactions (Impront II reverse transcription system; Promega, cat. no. M7112) were carried out according to the manufacturer's instructions. Oct4 was amplified with the primers shown in table 1 and GoTaq green master mix (Promega; cat. no. M7112) as follows: 1 cycle at 95° C. for 3 minutes, followed by 25 cycles of 30 seconds at 95° C.-60° C.-72° C., followed by one extension of 10 minutes at 72° C. In addition, quantitative PCR for Oct4 (cat. no. Mm658129 gH part no. 433182) and control GAPDH (probe mouse 4352339E0-611010) both from Applied Biosystems, were carried out with 2×TAQMAN Universal master mix (Applied Biosystems). DeltaCT values were determined according to user bulletin #2, ABI PRISM 7700, Dec. 11, 1997). All experiments were carried out with both heterozygous and homozygous ES cell lines and yielded essentially the same results.

### Inducible Caspase-3 Cell Line Experiments

We used a constitutively active caspase-3 gene (Casp3rev) as described in (Srinivasula et al., 1998) and a mutated version of Casp3rev (C1635) as described in (Kamada et al., 1998). These active and inactive forms of caspase-3 were derived from the plasmids pGEMCasp3rev (6730 bp) and pGEM MutCas3rev (3867 bp). The caspase-3 inserts were cut out of their backbone vectors with EcoRI and the insert (874 bp) cloned by blunt end ligation into the pLoxP2 vector and digested with XhoI-SmaI (3549 bp). The vector was co-electroporated with pSalk-Cre into 30×10⁶ A172 cells (960 μF, 25 millisec pulse. After electroporation, the cells were selected with G418 (400 μg/ml) and individual clones were picked and subsequently expanded. For the differentiation experiment, cells were plated into 6-well plates (1×10⁵/well) and stimulated with 1 μg/ml doxycycline (Sigma, cat. no. D9891) for 36 hours. They were then harvested, and Western blots for Oct4 (H-134 Santa Cruz, cat. no. SC9081), Nanog (Abeam, cat. no. AB14959; Chemicon 1/1000) and beta-tubulin (D10; Santa Cruz, cat. no. SC5274) performed. Secondary antibodies dilutions 1/5000 and 1/10000 consist of: Alexa-680 anti-rabbit (Molecular Probes, cat. no. A21077) or IRday 800 anti-rabbit (Rockland, cat. no. 611-131-121). The signal was detected with the Odyssey system.

### Detection of Nanog Cleavage by Caspase-3 In Vitro

Human Nanog was obtained from ATCC (cDNA clone cat. no. 10806397; Image Clone IDm no. 40004923; Gene Bank ID no. BC09827) and subcloned into pCR-BlendII-TOPO (Invitrogen). The mutation D69E was introduced into human hNanog with a site-directed mutagenesis kit (Quickchange II, Stratagene). Sequencing of both clones confirmed the identity of the human Nanog cDNA wild type (TOPOT-Nanog) and D69E mutation (TOPO-mut-Nanog). In vitro transcription and translation were performed according to the manufacturer's protocol (Promega, cat. no. L520A). TOPO-Nanog and TOPO-D69E-Nanog plasmid 1 μg was incubated at 30° C. with T7 quick master mix and 1 mM methionine plus biotin-tRNA (Promega, cat. no. L506A) in a total volume of 50 μl. The reaction was stopped after 90 minutes, and aliquots frozen at −80° C. Biotin labeled human hNanog and D69E-Nanog were detected after transfer to nitrocellulose membranes with streptavidin Alexa fluor 680—(Molecular Probes, cat. no. S21378, dilution 1/10000). Protein was digested with 40 ng recombinant active caspase-3 (BD Bioscience, cat. no. 51-66281V) in a total volume of 15 μl at pH 7.5 (6 mM Tris-Cl, 1.2 mM CaCl₂; 1 mM KCl, 5 mM DTT; 1.5 mM MgCl₂) (Laugwitz et al., 2001). The reaction product was run on 4-20% gradient PAGE gels (Bio-Rad), and protein was detected as previously described (streptavidin, Alexa Fluor 680 Odyssey system, LICOR).

### Detection of Nanog Cleavage by Endogenous Caspase-3

Nanog and D69E-Nanog were cloned into the pEF1-luciferase-IRES-NEO vector (kindly provided by David Spencer, Baylor College of Medicine) by using XhoI and XbaI cloning sites, which together with a FLAG tag were added at the C-terminus by a PCR-based strategy with 5′-ggactcgagatgagtgtggatccagcttgtcc-3′ (SEQ ID NO. 11) and 5′-tcctctagatcacttatcgtcatccttgtaatc-3′ (SEQ ID NO. 12) primers. The plasmid was transfected into 293 cells and R1 ES cells with lipofectamine 2000 (Invitrogen). R1 ES cells (1×10⁵) were plated into 6-well plates and transfected on the following day with 10 μl lipofectamine 2000 plus (Invitrogen, cat. no. 52887) with 4 μg plasmid DNA. Differentiation was induced with RA differentiation medium. Proteins were extracted with CHAPS buffer with two cycles of freezing (dry-ice) for 30 minutes and sonication with 20 pulses. Twenty μg of protein was subjected to PAGE (12% Bio-Rad, 2 hours at 80V) and transferred with TG buffer (Bio-Rad) supplemented with 0.05% SDS and 20% methanol to 0.2 μm nitrocellulose membranes (Bio-Rad). Detection was carried out with the FLAG antibody M2 (Stratagene, cat. no. 200470-21) in a 1/10,000 dilution in blocking buffer (Li—COR, cat. no. 927-4000). Equal loading was confirmed by probinding for alpha-tubulin (antibody D10, Santa Cruz, S.C. 5274).

### Nanog-Inducible ES Cell Line

Nanog and mutated D69E cDNAs were obtained by EcoRI digestion of TOPO-Nanog and TOPO-mut-Nanog plasmids, as described previously. The inserts (1263 bp each) were filled with Klenow and ligated to the p2LoxP vector which was cut with SmaI and XhoI and klenowed. The vector was co-electroporated with pSalk-Cre into A2lox ES cells (960 μF 220 millisecond pulse, 15×10⁶ cells). After electroporation, the cells were selected with 400 μg/ml of G418 (Invitrogen) and individual clones were picked, and subsequently expanded. Expression of inducible Nanog and inducible D69E Nanog was confirmed by western blotting and RT-PCR.

### Nanog Proliferation Assay

Nanog and D69E-Nanog clones were plated at a density of 5000 cells into 96 well plates, incubated overnight and then stimulated with RA for specified times. Cells were then pulsed with methyl-³H thymidine (Perkin Elmer, NET027) for 3 hours, harvested (Packard Filtermate harvester) and scintillation counts were measured with the Packard Topocount-NXT Microplate Scintillation and Luminescence Counter.

### Nanog Competition Assay

The inducible cell lines Nanog, D69E mutated Nanog, EGFP, and control A 172 were plated and incubated over night with 1 μM doxycycline. The Nanog inducible, D69E Nanog and A172 ES cell lines were mixed with the EGFP inducible cell line in a ratio of 20:80 and plated on the next day. Cells were maintained in ES cell medium without LIF. The percentage of EGFP-positive cells was determined with a FACScan instrument (Becton Dickinson) on days 0, 4, 8, 12, and 16 days after plating.

The results of the experiments presented in this Example are now described.

### Example 1

### Caspase Activity Increases after Induction of ES Cell Differentiation

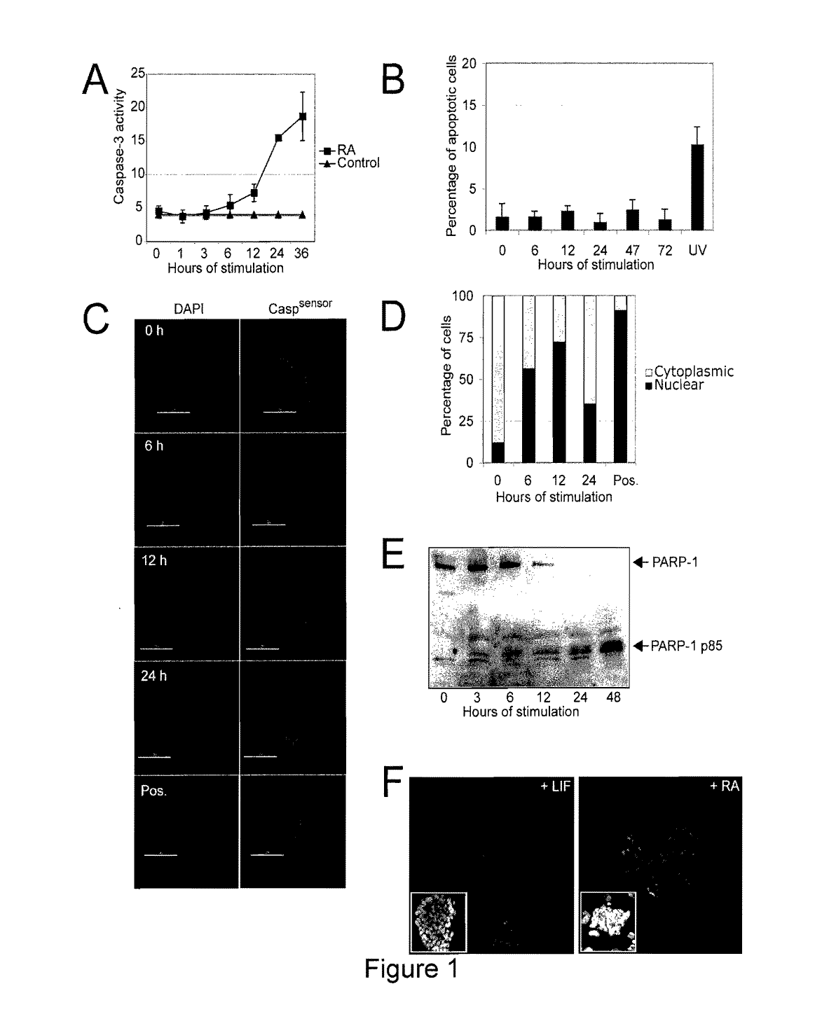

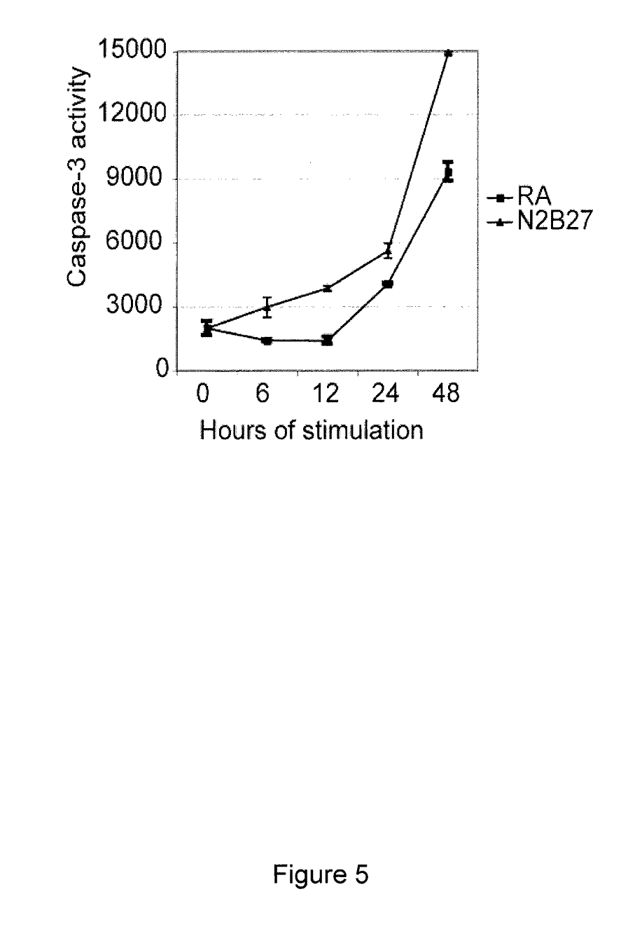

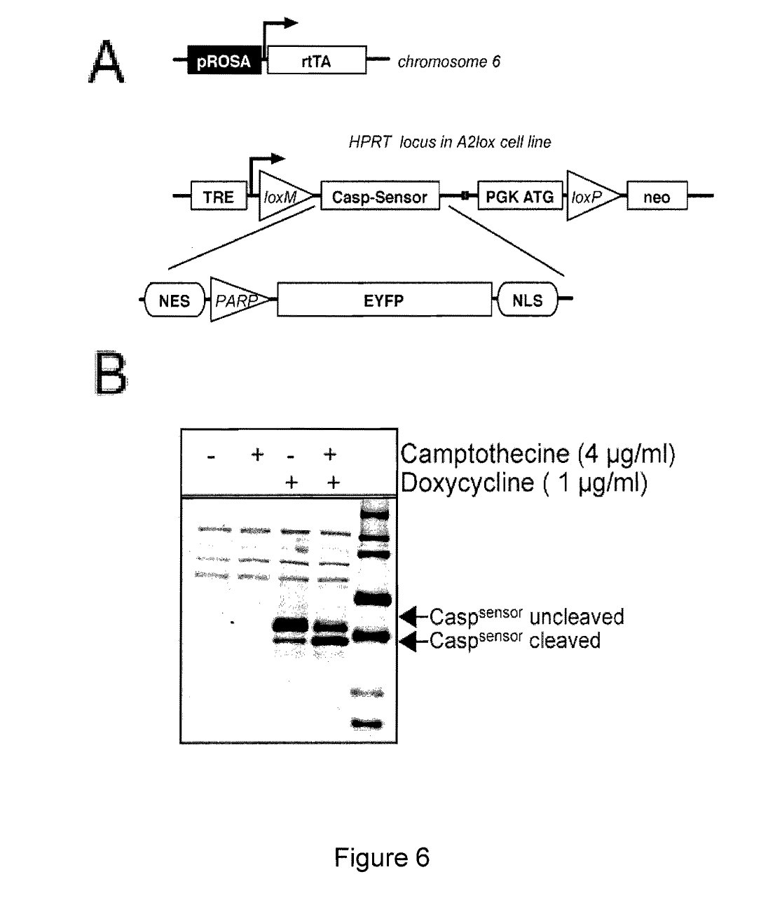

The presence of caspase activity in differentiating mouse ES cell cultures was tested. As shown in FIG. 1A and FIG. 5, caspase activity began to increase very shortly after the ES cells were stimulated with retinoic acid (RA) or plated in differentiation medium. To exclude an effect from increased apoptosis, the cultures were assayed for the percentage of cells undergoing apoptosis, demonstrating essentially no increases in this end point over 72 hours post-stimulation with RA (FIG. 1B). To substantiate that the caspase activity peaks were associated with cell differentiation and not programmed cell death, a caspase activity reporter cell line (Casp^{sensor} FIG. 6A) was generated in which enhanced yellow fluorescent protein (EYFP) could be seen in the cytoplasm as long as caspase activity was low or absent, but appeared in the nucleus when caspases were active (FIG. 1C). In their undifferentiated state, the ES cells showed mainly cytoplasmic EYFP staining, but upon induction of differentiation the EYFP signal shifted to the nucleus in most or an increased percentage of the ES cells (FIG. 1C-D), indicating the presence of caspase activity. Importantly, none of the caspase-positive cells appeared to be undergoing programmed cell death, as they lacked the classical features of nuclear condensation, nuclear fragmentation and membrane blebbing (not shown). Western blot analysis showed marked differences in the sizes of the EYFPs, indicating that the reporter protein had indeed been cleaved by caspases (FIG. 6B). The slight discrepancy in the kinetics of caspase activity shown in FIGS. 1A and 1D is attributable to the different cell densities required for the respective assays. Finally, it was asked if PARP-1, a recognized caspase target during the execution of apoptosis (Lazebnik et al., 1994), might also be cleaved after induction of differentiation. Western blot analysis and immunofluorescence microscopy with PARP-1 antibodies revealed what appeared to be cleaved PARP-1 (the p85 fragment) at 48 hours post-stimulation of ES cells (FIG. 1E-F), suggesting that as in cells undergoing apoptosis, PARP-1 was also cleaved by caspases during differentiation. Thus, differentiating ES cells show increased caspase activity that is not associated with programmed cell death.

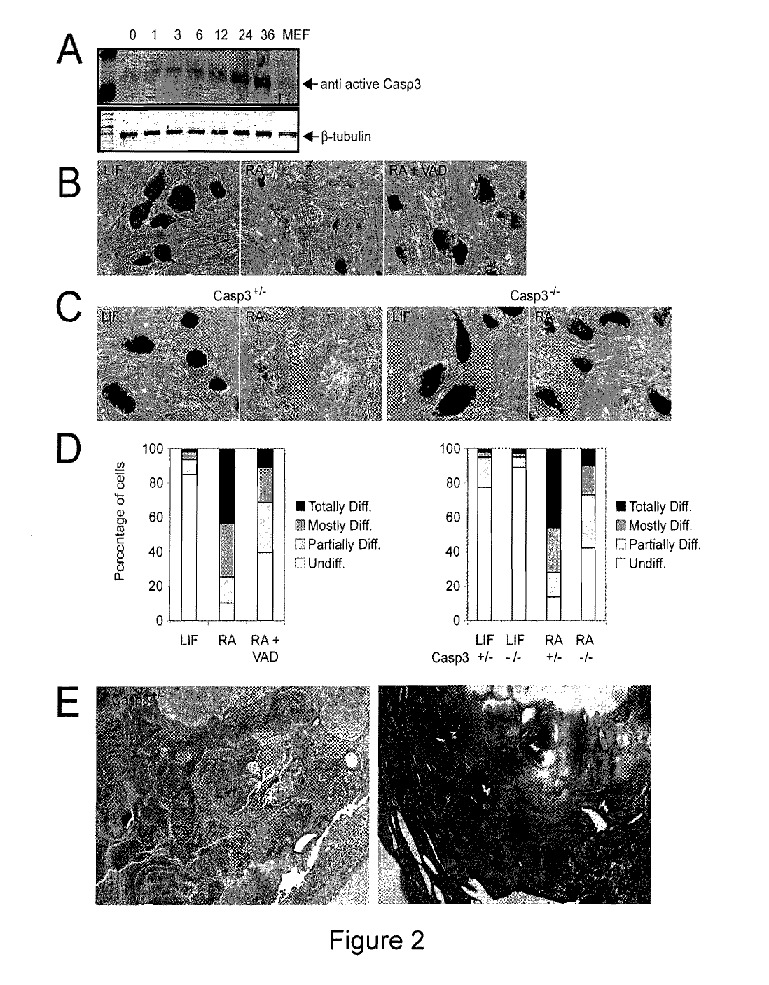

Because a variety of caspases could have accounted for the increases in caspase activity seen in our differentiating ES cell cultures, Western blot analysis was performed for active caspases in protein lysates from these cultures. Using antibodies against five major caspases, active caspase-3 was identified in the lysates (FIG. 2A) but other cysteine proteases were not detected (results not shown).

To demonstrate the functional relevance of caspase-3 to ES cell differentiation, the cells were first exposed to the caspase-blocking peptide VAD and differentiation induced with RA. This treatment clearly inhibited ES cell differentiation, although the block was not complete (FIG. 2B and FIG. 2D, left). Thus, while important for differentiation, caspase-3 activity may not have been the sole factor contributing to this process. Alternatively, the VAD peptide may not have fully inactivated the protease.

### Example 2

### Caspase-3 Knockout ES Cells Show a Differentiation Defect

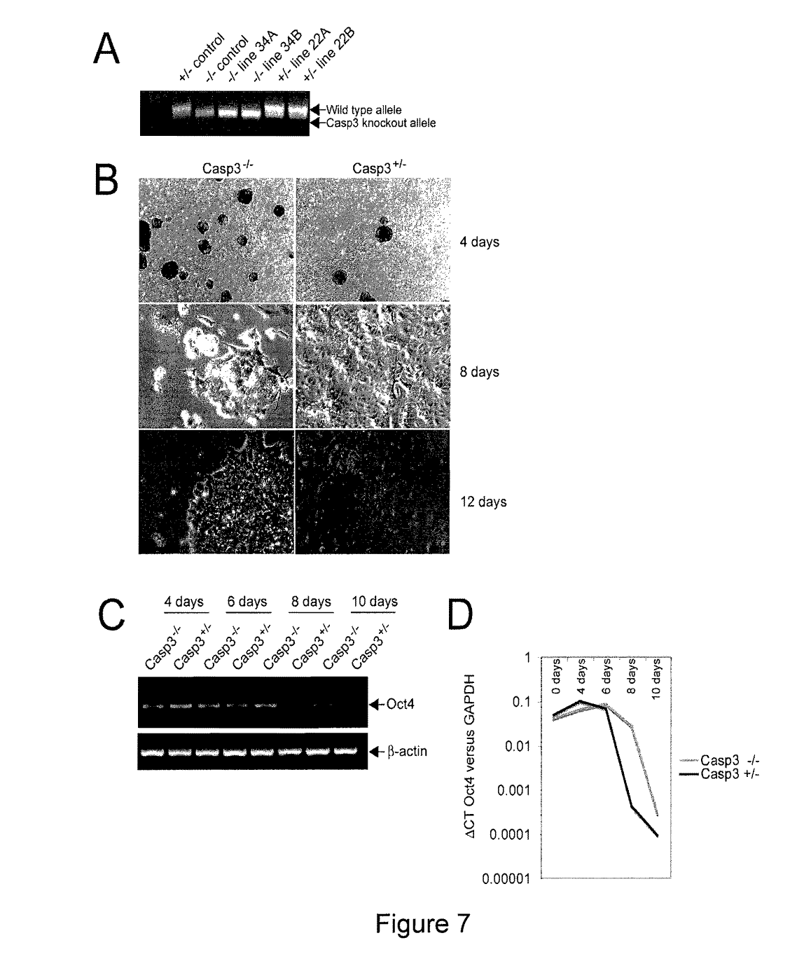

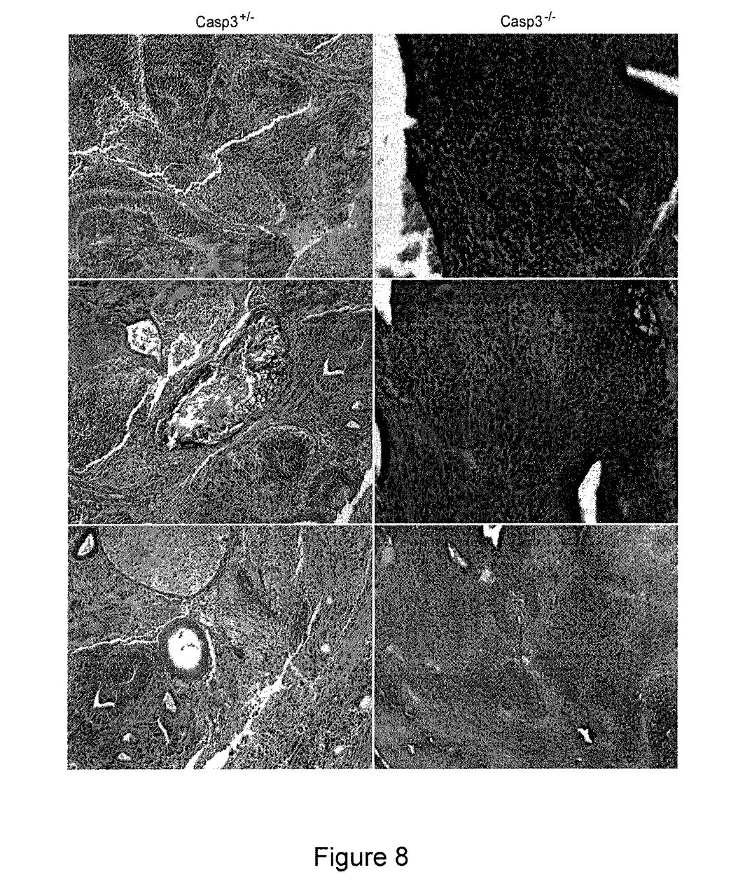

To substantiate a requirement for caspase-3 activity in ES cell differentiation, both homozygous and heterozygous lines of Casp3 knockout ES cells were generated (FIG. 7A). Deletion of the Casp3 locus lacked any discernible effect in undifferentiated ES cells; however, when exposed to RA, the Casp3^{-/-} ES cells showed an obvious delay in differentiation compared with Casp3^{+/-} cells (FIG. 2C, D, right). Similar results were obtained when the ES cells were induced to differentiate as embryoid bodies (FIG. 7B). As in the caspase blocking experiment, more than 25% of the Casp3^{-/-} ES cell colonies showed appreciable signs of differentiation after 5 days, while a substantial proportion of the colonies remained either completely undifferentiated (42%) or only partially differentiated (31%). Quantification of Oct4 expression in Casp3^{-/-} ES cells by PCR (FIG. 7C and FIG. 7D) showed a reduction in this transcription factor with time after differentiation, reinforcing the idea that a differentiation delay is associated with the absence of this protease in ES cells. Casp3^{+/-} and Casp3^{-/-} ES cells were also injected into immunocompromised mice, observing tumor formation at the injection sites 10-14 days later in both experimental groups. Histological examination of the tumors revealed differentiated cells from all germ layers in mice injected with the Casp3^{+/-} ES cells, in contrast to the mainly undifferentiated or immature cells in Casp3^{-/-} ES cells (FIG. 2E and FIG. 8). Taken together, the caspase-blocking and Casp3 knockout data support direct involvement of caspase-3 in ES cell differentiation.

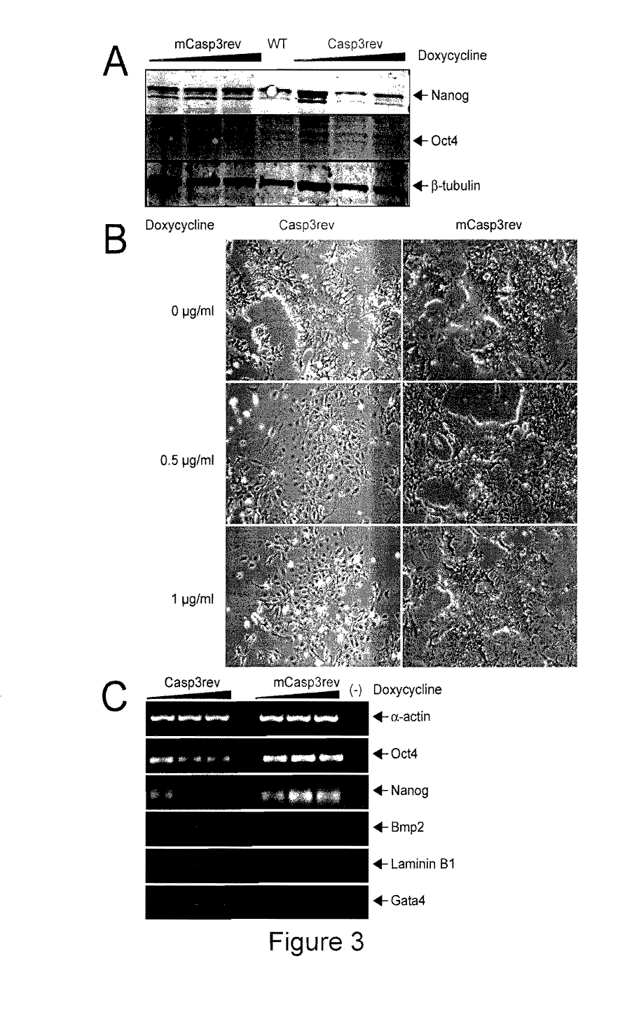

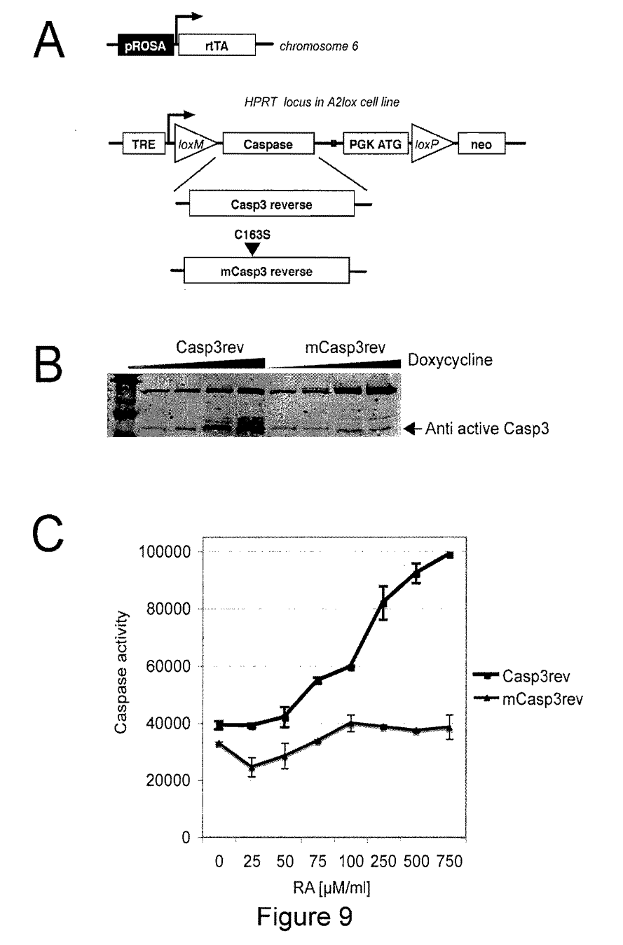

If caspase-3 indeed promotes the differentiation of ES cells, it should be possible to demonstrate this effect by modulating levels of the active protease. Targeted insertions of cDNAs encoding a constitutively active (Casp3rev) or mutated form of caspase-3 (mCasp3rev) were made upstream of the HPRT locus of A2lox ES cells; these cDNAs were under the control of a tetracycline-inducible promoter (FIG. 9A). Induction of higher levels of inducible caspase-3 with doxycycline in ES cells stimulated differentiation, coincident with a reduction of the Nanog and Oct4 transcription factors, an increase in the expression of miscellaneous differentiation factors, and an obvious change in cell morphology associated with differentiation (FIG. 3A, B, C). Whether such stimulation favors differentiation to a particular cell type (e.g., endoderm) or simply releases ES cells from the self-renewal machinery to be stimulated by other extrinsic or intrinsic signals required for cell fate commitment is unclear.

### Example 3

### Nanog is Cleaved Upon Induction of Differentiation

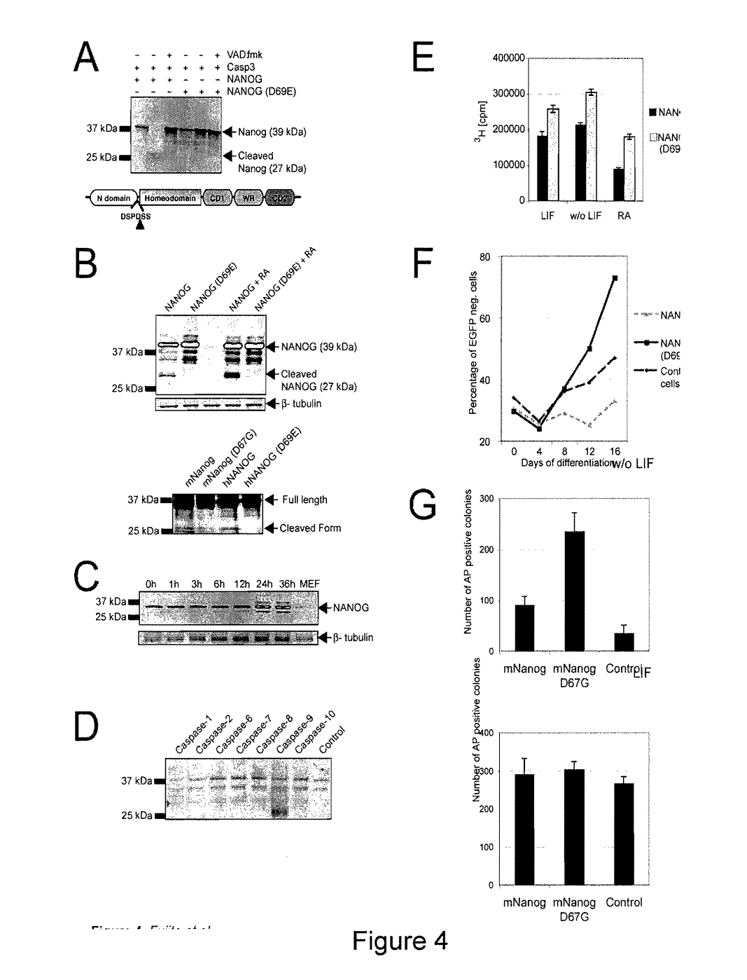

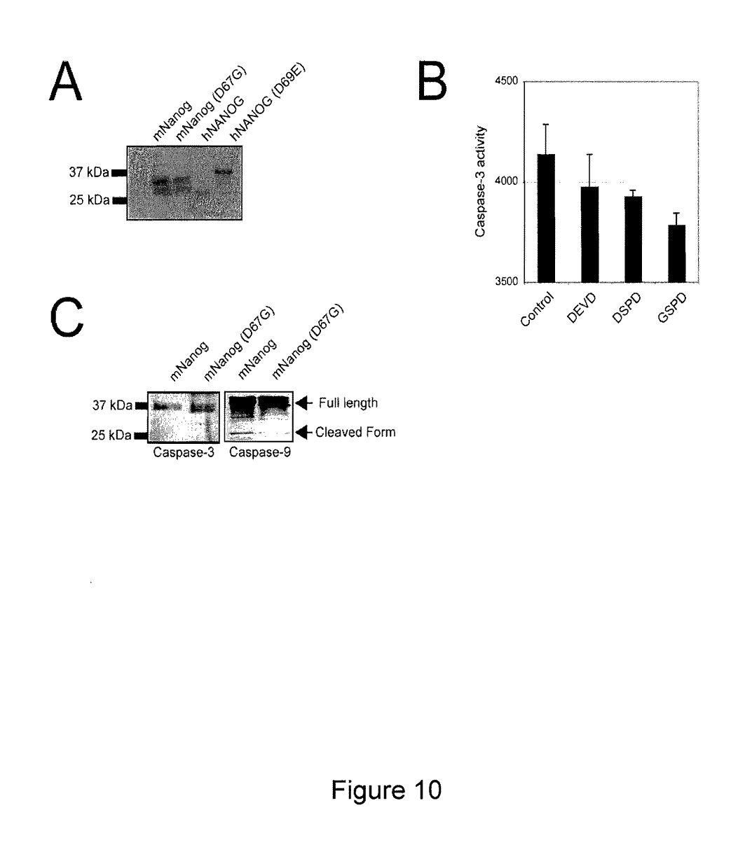

To identify the differentiation-specific molecular targets of caspase-3 in mouse ES cells, an in vitro caspase-3 cleavage assay was developed and used to determine the cleavage of Sox2, Oct4 and Nanog transcription factors, all of which maintain ES cells in a self-renewing state. Caspase-3 cleaved human (h) Nanog in vitro (FIG. 4A), while Sox2 and Oct4 remained uncleaved (data not shown). Examination of the hNanog amino acid sequence revealed conserved residues at position 69 and at position 70 between the N8 terminal transcriptional transactivator (Pan and Pei, 2003, Cell Res. 13:499-502) and the homeodomain (FIG. 4A), that likely serve as the caspase-3 cleavage site. Indeed, when a single amino acid in this putative recognition sequence was mutated (yielding D69E hNanog), caspase-3 was no longer able to cleave the protein in vitro (FIG. 4A). It was also noted that mouse (m) Nanog has a single amino acid substitution (D->G) at position 64, that is 3 bases upstream of the caspase-3 cleavage site, a modification that could decrease the ability of caspase-3 to cleave mNanog. An in vitro caspase-3 substrate affinity assay revealed that the cleavage site in mNanog (GSPD) was still targeted by caspase-3 (FIG. 10B). However, after performing the same in vitro cleavage assays as used with hNanog, very little or no cleavage of recombinant mNanog by caspase-3 was observed (FIG. 10A). To determine whether both hNanog and mNanog and their corresponding mutants are cleaved in differentiating ES cells in vivo, all four constructs were studied in ES cells treated with RA (FIG. 4B). Western blot analysis revealed cleavage of both wild-type hNanog and wild-type mNanog, but not their mutant forms harboring modified caspase cleavage sites (aa 67-68 in mNanog and aa 69-70 in hNanog) (FIG. 4B). In addition, Western blot analysis with antibodies against endogenous Nanog detected a smaller band at 24 and 36 hours post-induction that corresponded to the cleaved form of Nanog (FIG. 4C). Thus, given the apparently reduced activity of caspase-3 toward mNanog in vitro, compared with its effective cleavage of this transcription factor in vivo, another, caspase-3-dependent protease may function with caspase-3 to modify the regulatory activity of mNanog. By screening a panel of caspases for their ability to cleave mNanog in vitro (FIG. 4D, left), caspase-9 was identified as the most likely candidate for this role (FIG. 4D and Figure S6C). Indeed, the activity of caspase-9 can be drastically enhanced either by active caspase-3 (Zou et al. 2003, J. Biol. Chem. 278:8092-8098) or by Procaspase-3 (Yin et al., 2006, Mol. Cell. 22:259-268) supporting an important contribution of caspase-9 to Nanog cleavage in ES cells.

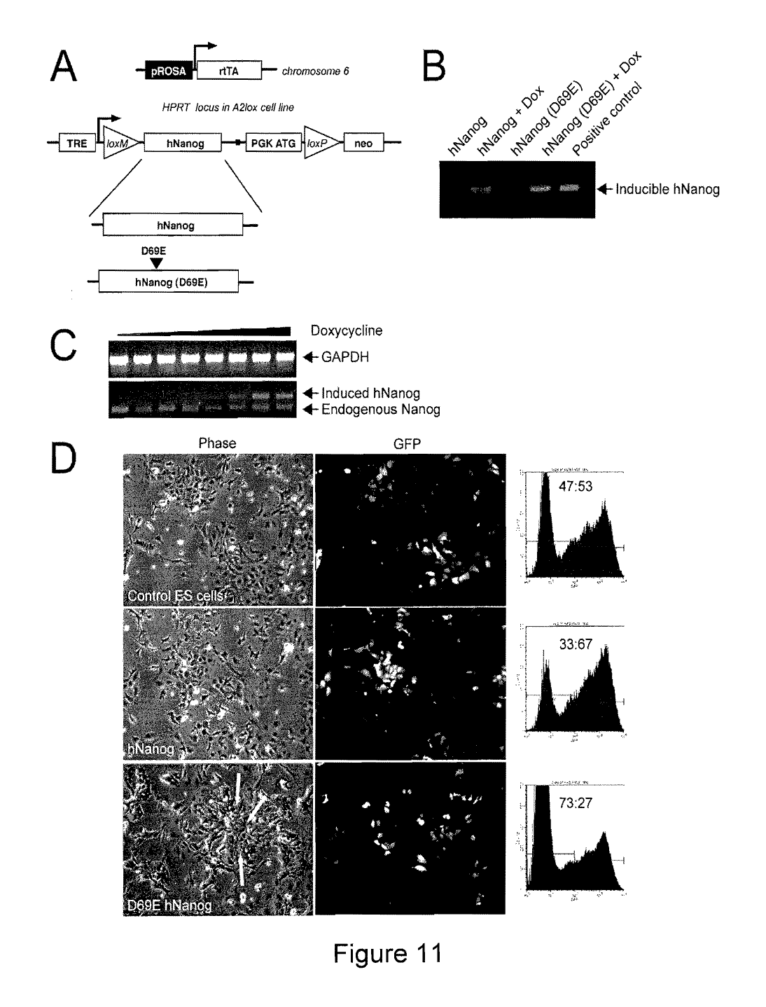

If Nanog is indeed one of the principal targets of caspases in ES cells, its cleavage should have profound effects on whether the cells remain in a state of pluripotency or differentiate. To test this prediction, ES cells were generated harboring cDNA coding for hNanog or D69E, the caspase cleavage-resistant form of hNanog targeted upstream of the HPRT locus under the control of a tetracycline-inducible element (FIG. 11A and FIG. 11B). Importantly, the expression of induced hNanog reached levels comparable to those of endogenous Nanog (FIG. 11C). ES cells expressing the D69E form of hNanog had a clear proliferative advantage over cells with the wild-type allele when cultured under conditions that promote differentiation (FIG. 4E, F). They also lacked evidence of morphological changes, in contrast to ES cells carrying the wild-type form of Nanog (FIG. 11D). To assess the antidifferentiation effects of caspase cleavage mNanog ES cells transfected with wild-type and D67G (cleavage-resistant) mNanog were plated at clonal densities and colony formation was analyzed 3 days later. The vast majority of colonies expressing the cleavage-resistant form of mNanog appeared morphologically unaffected and were positive for alkaline phosphatase, whereas a significantly lower number of colonies expressing wild-type mNanog consisted of undifferentiated, alkaline phosphatase-positive cells (FIG. 4G). Thus, caspase mediated cleavage of Nanog promotes ES cell differentiation.

### Example 4

### ES Cells Exploit Caspases for Rapid and Specific Deactivation of Nanog to Preserve Pluripotency

The data presented herein indicate that ES cells exploit caspases for rapid and specific deactivation of Nanog, thus disrupting the autoregulatory circuit that otherwise preserves pluripotency in these cells. They indicate further that caspase-3 plays a dominant role in this negative regulation, by acting directly on Nanog or interacting as a cofactor with caspase-9, which then deactivates the transcription factor. The action of caspases on Nanog appears to separate the N-terminal domain from the homeodomain, leading to the destabilization and subsequent degradation of the protein. From our experiments it is clear that both human and mouse Nanog are cleaved by caspases, and therefore it is likely that both of them are completely interchangeable with regard to caspase-mediated cleavage during the differentiation of ES cells. That the caspase cleavage-resistant forms of Nanog have a significantly stronger anti-differentiation effect in ES cells than does wild-type Nanog firmly suggests that caspase-mediated cleavage of endogenous Nanog plays a critical role in ES cell differentiation.

The novel link identified herein between ES cell differentiation and programmed cell death helps to explain several poorly understood observations on these ostensibly distinct processes, both in vitro and in the early embryo. For instance, some components of the cell death system, such as Bcl-2, protect ES cells not only from apoptosis but also from differentiation (Yamane et al., 2005, PNAS 102:3312-3317), while p53 has been shown to be directly involved in both the control of cell proliferation and apoptosis and the differentiation of ES cells (Lin et al., 2005, Nature Cell Biol. 7:165-171). Moreover, other proteolytic components of ES cells, such as the proteasome, appear to have direct roles in the control of stem cell self-renewal (Szutorisz et al., 2006, Cell 127:1375-1388). Future studies will need to address the question of whether caspase activity indeed actively promotes differentiation, as our data suggest, or perhaps functions as part of a mechanism for the selective elimination of undifferentiated cells. In either case, the net outcome—cell differentiation—would be the same.

Selective targeting of Nanog by caspases is consistent with evidence implicating this transcription factor as a “master” regulator of the pluripotent state (Chambers et al., 2003, Cell 113:643-655; Ivanova et al., 2006, Nature 442:533-538; Mitsui et al., 2003, Cell 113:631-642). However, given the remarkable complexity of the pathways controlling cell death and differentiation, it is likely that caspase-3, acting alone or in collaboration with caspase-9, as well as other mediators of cell death, work by targeting pluripotency factors in addition to Nanog. This hypothesis is supported by the absence of any overt phenotype in the pre-implantation embryos of Casp3 knockout mice (Kuida et al., 1996, Nature 384:368-372; Woo et al., 1998, Genes Dev. 12:806-819).

The data presented herein leave unanswered the major question of why caspase-3 activation during differentiation is limited and does not self-amplify as seen during apoptosis. Potential mechanisms for such control of caspase activity include subcellular localization of the enzyme, the caspase activity level itself, phosphorylation or other forms of structural modification, as well as direct or indirect interaction with protein inhibitors, such as IAPs. Given that ES cells carrying the cleavage-resistant form of Nanog proliferate better than those with the wild-type allele, it seems reasonable to suggest that the cleaved form of Nanog might exert a dominant-negative effect on some of the normal functions of Nanog.

A more comprehensive understanding of the molecular pathways controlling ES cell self-renewal and differentiation, in particular the apparent molecular link between programmed cell death and cell differentiation, would not only accelerate efforts to generate clinically relevant cell types from ES cells, but may also facilitate the

reprogramming of differentiated cells to enter a pluripotent state (Okita et al., 2007, Nature 448:313-317; Silva et al., 2006, Nature 441:997-1001), for example, by blocking caspase activity. The affinity of caspase-3 and caspase-9 for the transcription factor Nanog may be a paradigm for other potential caspase targets in ES cells; hence, it should be possible to exploit the caspases in experimental screens to identify other factors that regulate stem cell pluripotency. Finally, the involvement of caspases in non-apoptotic pathways, as demonstrated here and elsewhere (Arama et al., 2003, Cev. Cell 4:687-697; De Bolton et al., 2002, Blood 100:1310-1317; De Maria et al., 1999, Blood 93:796-803; De Maria et al., 1999, Nature 401:489-493; Ishizaki et al., 1998, J. Cell Biol. 140:153-158), suggests that efforts to block apoptosis via caspase inhibition for therapeutic purposes may have much broader implications than initially thought, especially for stem cells.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed:

1. A method of maintaining the pluripotency of an embryonic stem (ES) cell, said method comprising inhibiting the cleavage of Nanog in said cell, by contacting said cell with a caspase inhibitor, wherein when said cell is contacted with said inhibitor, said cell remains pluripotent.

2. The method of claim 1 wherein said inhibitor is selected from the list consisting of an antibody, an siRNA, a ribozyme, an oligonucleotide, a peptide, and a small molecule.

3. The method of claim 1, wherein said caspase is selected from the group consisting of caspase-3 and caspase-9.

4. The method of claim 1, wherein said ES cell is a human, ES cell.

5. A method of inhibiting caspase activity in an ES cell, said method comprising contacting said cell with a caspase inhibitor, wherein when said cell is contacted with said inhibitor, the activity, stability, or expression of said caspase in said cell is inhibited, attenuated, or blocked.

6. The method of claim 5, wherein said inhibitor is selected from the group consisting of an antibody, an siRNA, a ribozyme, an oligonucleotide, a peptide, and a small molecule.

7. The method of claim 5, wherein said caspase is selected from the list consisting of caspase-3 and caspase-9.

8. The method of claim 5, wherein said ES cell is a human, ES cell.

9. A method of preventing differentiation of an ES cell, said method comprising contacting said cell with a caspase inhibitor, wherein when said cell is contacted with said inhibitor said cell does not differentiate.

10. The method of claim 9, wherein said inhibitor is selected from the group consisting of an antibody, an siRNA, a ribozyme, an oligonucleotide, a peptide, and a small molecule.

11. The method of claim 9, wherein said caspase is selected from the list consisting of caspase-3 and caspase-9.

12. The method of claim 9, wherein said ES cell is a human, ES cell.

13. A method of maintaining the pluripotency of an embryonic stem (ES) cell, said method comprising, providing said cell with an effective amount of cleavage-resistant Nanog.

14. The method of claim 13, wherein said cleavage-resistant Nanog is not expressed by said cell and is provided to said cell exogenously.

15. The method of claim 13, wherein said cleavage-resistant Nanog is expressed by said ES cell.

16. The method of claim 13, wherein said ES cell is a human, ES cell.

17. A method of initiating differentiation of an ES cell, said method comprising the steps of:
a) maintaining the pluripotency of an ES cell by contacting said cell with a caspase inhibitor, wherein when said cell is contacted with said inhibitor, said cell remains pluripotent;
b) preventing said caspase inhibitor from contacting said cell by removing said inhibitor, inhibiting said inhibitor, or antagonizing the effects of said inhibitor, whereby the expression, activity, or stability of said caspase in said cell is enhanced, and further whereby said cell initiates differentiation.

18. The method of claim 17, wherein said ES cell is a human, ES cell.


SEQUENCE LISTING (12 sequences)

SEQ ID NO: 1 - DNA, 28 bp, Artificial Sequence
  Chemically synthesized
gatgtcgactcagatccgctagccgcca

SEQ ID NO: 2 - DNA, 28 bp, Artificial Sequence
  Chemically synthesized
gagtactttatctagatccggtggatcc

SEQ ID NO: 3 - DNA, 20 bp, Artificial Sequence
  Chemically Synthesized
tgtcatctcgctctggtacg

SEQ ID NO: 4 - DNA, 20 bp, Artificial Sequence
  Chemically synthesized
ccctttctgcctgtcttctg

SEQ ID NO: 5 - DNA, 20 bp, Artificial Sequence
  Chemically synthesized
agacaatcggctgctctgat

SEQ ID NO: 6 - DNA, 20 bp, Artificial Sequence
  Chemically synthesized
atactttctcggcaggagca

SEQ ID NO: 7 - DNA, 24 bp, Artificial Sequence
  Chemically synthesized
ggcgttctctttggaaaggtgttc

SEQ ID NO: 8 - DNA, 20 bp, Artificial Sequence
  Chemically synthesized
ctcgaaccacatccttctct

SEQ ID NO: 9 - DNA, 23 bp, Artificial Sequence
  Chemically synthesized
ggcccagagcaagagaggtatcc

SEQ ID NO: 10 - DNA, 21 bp, Artificial Sequence
  Chemically synthesized
acgcacgatttccctctcagc

SEQ ID NO: 11 - DNA, 32 bp, Artificial Sequence
  Chemically synthesized
ggactcgagatgagtgtggatccagcttgtcc

SEQ ID NO: 12 - DNA, 33 bp, Artificial Sequence
  Chemically synthesized
tcctctagatcacttatcgtcatccttgtaatc